(19) United States
(12) Patent Application Publication
(10) Pub. No.: US 2015/0191778 A1
(43) Pub. Date: Jul. 9, 2015

(54) COMPOSITIONS AND METHODS FOR DETECTING AND IDENTIFYING SALMONELLA ENTERICA STRAINS

(71) Applicant: LIFE TECHNOLOGIES CORPORATION, CARLSBAD, CA (US)

(72) Inventors: CRAIG CUMMINGS, PACIFICA, CA (US); ELENA BOLCHAKOVA, UNION CITY, CA (US); MANOHAR FURTADO, SAN RAMON, CA (US)

(21) Appl. No.: 14/594,397

(22) Filed: Jan. 12, 2015

Related U.S. Application Data
(63) Continuation of application No. 13/451,479, filed on Apr. 19, 2012, now abandoned.
(60) Provisional application No. 61/477,142, filed on Apr. 19, 2011.

(51) Int. Cl. C12Q 1/68; C12N 15/10

(52) U.S. Cl. CPC C12Q 1/689; C12N 15/1089; C12Q 2600/156; C12Q 2600/158

(57) ABSTRACT

The present specification describes several novel SNPs of Salmonella enterica subsp. enterica. SNP profiles comprising allelic compositions at each SNP position are described which may be used to identify and differentiate different strains and serovars of Salmonella enterica subsp. enterica. The specification also describes several compositions, methods and kits useful for identifying and differentially distinguishing strains and serovars of Salmonella enterica subsp. enterica.

### CROSS-REFERENCE TO RELATED APPLICATIONS

This application is a continuation of U.S. patent application Ser. No. 13/451,479 filed Apr. 19, 2012, which application claims priority to U.S. Provisional Patent Application Ser. No. 61/477,142, filed Apr. 19, 2011, the entire contents of which are incorporated herein by reference in their entirety.

### SEQUENCE LISTING

The instant application contains a Sequence Listing which was submitted in ASCII format via EFS-Web and is hereby incorporated by reference in its entirety. Said ASCII copy, created on Apr. 12, 2012, is named LT00497.txt and is 6,160,501 bytes in size.

### FIELD

The present specification relates, in some embodiments, to compositions, methods and kits for detection and identification of Salmonella enterica subsp. enterica strains and serovars (serological variants). In some embodiments, the disclosure describes several novel single nucleotide polymorphisms (SNPs) and compositions derived therefrom (including probes and primers), which may be used in methods of the disclosure to detect and/or identify a Salmonella enterica subsp. enterica strain from a sample and in some embodiments to identify the serotype of a S. enterica subsp. enterica.

### BACKGROUND

S. enterica strains and serovars are common food borne microbes causing diseases in humans and in animals. Some S. enterica strains cause enteric (intestinal) infections, often referred to as salmonellosis. Other Salmonella enterica strains such as Salmonella Typhi and S. Paratyphi cause typhoid fever.

Traditional serotyping, the standard method for characterization of Salmonella enterica serotypes, is laborious and time consuming, and requires the maintenance of large panels of specific antisera, which is feasible for only a small number of large microbiology reference labs.

Traditional serotyping is also unable to differentiate between evolutionarily distinct subgroups that often exist within a single polypyletic serotype.

### SUMMARY

The present specification relates in some embodiments to identification of several novel single nucleotide polymorphisms (SNPs). These SNPs may be used to differentially identify S. enterica subsp. enterica strains and serovars. In some embodiments, one or more SNP's identified herein may be used to differentiate between closely related strains and serovars of Salmonella enterica subsp. enterica.

The present disclosure in some embodiments provides at least 52 SNPs operable to identify Salmonella enterica subsp. enterica strains and serovars. The fifty two novel SNPs identified herein are comprised in nucleic acid sequences comprised in SEQ ID. NOs: 1-52 at position 101 in each of these sequences (see Table 2 attached at the end of the specification). The SNPs located at position 101 are shown in Table 2 by a lowercase nucleotide and correspond to a coordinate position (shown in column 1 of Table 2) in the genomic sequence of Salmonella Enteritidis (also referred to as Salmonella enterica subsp. enterica serovar Enteritidis) described in SEQ ID NO: 53 (which is also described in GenBank ID AM933172.1). SEQ ID NOs: 1-52 comprise SNPs of the disclosure at position 101 and are flanked by 100bp of genomic DNA on either side (3′ and 5′ side) of the SNP (coordinate positions of left and right flanking sequences in reference of the S. Enteritidis genome are shown in columns 2 and 3 of Table 2).

According to some embodiments, SNPs of the disclosure may be correlated to various Salmonella enterica serotypes and strains and an SNP profile database may be created. Some embodiments describe a computer readable medium used to store a SNP profile database of the disclosure. In some embodiments, a master SNP profile database may be created having all known SNP profiles. An SNP profile of a master database will have data, such as but not limited to, the composition of an SNP allele for each SNP position, and the correlation of SNP allelic compositions at different SNP locations with a serovar and/or a strain.

Assays and methods may be designed using a SNP profile database for analysis and identification including differential identification of Salmonella strains and/or serovars. For example, to determine the strain and/or serovar, a nucleic acid isolated from a Salmonella enterica containing sample, the sample nucleic acid may be tested to determine the allelic composition for at least ten SNPs selected from a larger panel of predetermined SNPs for which serovars have been correlated (such as for example, a master SNP profile database, which in one embodiment may comprise a profile database of the 52 panel of SNPs of the disclosure). The allelic composition of each SNP tested from the sample may then be stored in a sample nucleic acid SNP profile. The sample nucleic acid SNP profile may then be compared with the master SNP profile database to determine the correlation of SNPs and SNP allelic compositions to a particular Salmonella enterica strain and/or serovar. Determining the presence of certain alleles and certain allelic compositions identifies the strain or serovar of Salmonella enterica. Comparison of SNP profiles and correlation may be performed using a computer system or may be performed manually.

In other examples, to determine the strain and/or serovar, a nucleic acid isolated from a Salmonella enterica containing sample, the sample nucleic acid may be tested to determine the allelic composition for at least two, at least three, at least four, at least five, at least six, at least seven, at least eight, at least nine and/or at least ten SNPs selected from a larger panel of predetermined SNPs for which serovars have been correlated (such as for example, a master SNP profile database, which in one embodiment may comprise a profile database of the 52 panel of SNPs of the disclosure).

The present disclosure, in some embodiments, also provides compositions derived from the SNPs of the disclosure. Accordingly, in some embodiments, oligonucleotides comprising primers operable for amplifying (and identifying) one or more SNPs from the nucleic acid of a S. enterica strain are described. Exemplary primers comprise isolated nucleic acid sequences comprised in SEQ ID NOs: 54-105 and SEQ ID NOs: 106-157. However, primers of the disclosure are not limited to the sequences and oligonucleotides disclosed in SEQ ID NOs: 54-157, and one of skill in the art, in light of the present teachings will appreciate that additional primers are also disclosed by the present disclosure. For example, in some embodiments, isolated nucleic acid sequences of the disclosure may have at least 90% sequence identity, at least 80% sequence identity, and/or at least 70% sequence identity to nucleic acid sequences comprised in SEQ ID NOs: 54-157. Other primers may be designed that are to flank a SNP of the disclosure and to form an amplification product comprising an SNP.

In some embodiments, probes operable for identifying one or more SNPs from the nucleic acid of a S. enterica strain are provided. Exemplary probes are described in SEQ ID NOs: 158-209 which correspond to probes for one allele of a SNP, and SEQ ID NOs: 210-261, which correspond to example probes that may be used to identify the other allele of a SNP. In the probes described in SEQ ID NOs: 158-261 (see Table 3), the lowercase nucleotide corresponds to the SNP. However, probes of the disclosure are not limited to the sequences and nucleotides disclosed in SEQ ID NOs: 158-261, and one of skill in the art, in light of the present teachings will appreciate that additional probes are also disclosed by the present disclosure. For example, any nucleotide sequence having at least 10 nucleotides, at least 20 nucleotides, at least 30 nucleotides, or at least 40 nucleotides, comprising a SNP may be used as a probe. For example, in non-limiting examples, any nucleotide having at least nucleotides 100-102 of sequences described in SEQ ID NOs: 1-52 and at least 10 additional nucleotides on either the 5′ or the 3′ side of these sequences may be used as a probe of the disclosure. In other non-limiting examples, any nucleotide having at least nucleotides 100-102 of sequences described in SEQ ID NOs: 1-52 and at least 5 additional nucleotides on both the 5′ and the 3′ side of these sequences may be used as a probe of the disclosure. In yet other embodiments, probe sequences of the disclosure may have at least 90% sequence identity, at least 80% sequence identity, and/or at least 70% sequence identity to nucleic acid sequences comprised in SEQ ID NOs: 54-157.

The present disclosure, in some embodiments, describes methods for identifying Salmonella enterica strains and serovars based upon determining the allelic composition of one or more SNPs identified herein. In some embodiments, a method may comprise determining the allelic composition of a panel of at least 10 SNPs to identify and/or differentially detect a strain or a serovar of S. enterica. In some embodiments, a method may comprise determining the allelic composition of all the 52 SNPs to identify and/or differentially detect a strain or a serovar of S. enterica. In some embodiments, a method may comprise determining the allelic composition of a panel of at least 2, at least 3, at least 4, at least 5, at least 6, at least 7, at least 8, at least 9 and/or at least 10 SNPs to identify and/or differentially detect a strain or a serovar of S. enterica.

Determining the allelic compositions and/or genotyping and/or SNP detection may comprises one or more technologies such as but not limited to sequencing (also see the next sentence), amplification, hybridization, high throughput screening methods, bead-based liquid microarray platforms, mass spectrometry, nanostring, microfluidics, chemiluminescence, oligonucleotide ligation, enzyme technologies and combinations thereof. Determining the allelic compositions and/or genotyping and/or SNP detection by sequencing may comprises one or more technologies and/or platforms such as but not limited to genomic sequencing, sequencing targeted regions on CE or semiconductor platforms, multiplex sequencing of all SNP containing regions by CE or semiconductor sequencing and/or by combining with sequencing regions such as but not limited to rfb, fliC and fljB regions on the same platform. Molecular assays to detect SNPs may include amplification performed by a variety of methods such as but not limited to TaqMan®, SnapShot® and other high throughput screening methods know in the art in light of this specification.

Some methods for identifying and/or detecting S. enterica strains and/or serovars in a sample may comprise using an isolated nucleotide sequence composition of the disclosure for detection. Exemplary compositions of the disclosure used for detection methods may comprise, but are not limited to, SEQ ID NO: 54-157, and/or SEQ ID NO:158-261, fragments thereof, at least 10 contiguous nucleotide sequences thereof, complements thereof, isolated nucleic acid sequence comprising at least 90% nucleic acid sequence identity to the sequences set forth above and/or labeled derivatives thereof.

In some embodiments, methods of the disclosure may comprise: isolating a nucleic acid from a sample suspected of having a S. enterica strain and/or a sample from which one desires to detect and/or identify a specific S. enterica strain; amplifying one or more SNP comprising nucleic acid sequences (target nucleic acid sequence) from the nucleic acid from the sample to form an amplification product; and determining the allelic composition of the SNP comprised in the amplified product. Amplification may be repeated using a different set of primer pairs, each primer pair operable to hybridize to and amplify a target nucleic acid sequence comprising another SNP and determining the allelic composition of each SNP until the allelic composition of a panel of SNPs is determined. Once sufficient allelic composition is determined a correlation may be made to which serovar or strain of S. enterica the allelic composition may be assigned to. In some embodiments, at least 10 SNPs allelic compositions may be determined. In some embodiments, a panel of at least 10 SNPs selected from the SNPs comprised in SEQ ID NOs: 1-52 may be amplified and detected. In some embodiments, all SNPs comprised SEQ ID NOs: 1-52 may be amplified and detected to identify and/or type a strain of S. enterica. Amplification reactions may be multiplexed to detect a panel of SNPs.

In some embodiments, a pair of primers used for amplification may comprise the nucleic acid sequence of SEQ ID NO: 54-105, and/or SEQ ID NO: 106-157 and/or labeled derivatives thereof. For example, as shown in Table 3, a primer pair shown as reverse and forward primers may be used to amplify the corresponding SNP in the same row. Thus for example, a primer pair may comprise a first primer and a second primer, the first primer having a SEQ ID NO: 54 may be used as a forward primer, the second primer having a nucleic acid of SEQ ID NO: 106 may be used as a reverse primer may be used to amplify a SNP comprised in SEQ ID NO 1. Primers may be labeled and nucleic acid amplification products may be detected and/or identified by a variety of methods known in the art including but not limited to size analysis of an amplified product; sequencing an amplified product; hybridization with a probe; 5′ nuclease digestion; single-stranded conformation polymorphism; allele specific hybridization; primer specific extension; and oligonucleotide ligation assay.

In some embodiments, methods of the disclosure may comprise: isolating a nucleic acid from a sample suspected of having a S. enterica strain and/or a sample from which one desires to detect and/or identify a specific S. enterica strain; hybridizing one or more SNP comprising regions of the nucleic acid from the sample using one or more probes, each probe designed to bind specifically to a region comprising an SNP; and detecting the hybridized probe-nucleic acid complex. Probes used may be labeled to enable detection. Multiplex hybrid detection maybe enable by using differentially labeled probes. Other detection methods may comprise size analysis of the hybridized product; sequencing an amplified product; hybridization with a probe; 5′ nuclease digestion; single-stranded conformation polymorphism; and/or allele specific hybridization.

In some embodiments, probes used for hybridization and/or for detection of amplified products comprising a SNP may comprise the nucleic acid sequence of SEQ ID NO: 158-209, and/or SEQ ID NO: 210-261, and may comprise labeled derivatives thereof. For example, as shown in Table 3, probes 1 labeled with FAM-MGB and/or probes 2 labeled with VIC-MGB may be used to identify the corresponding SNP in the same S. Entritidis coordinate position (see Tables 2 and 3). Thus for example, probes having SEQ ID NO: 158 and SEQ ID NO: 210 may be used to hybridize to an SNP comprised in SEQ ID NO: 1. Each probe is operable to hybridize to one allelic composition of the SNP described in SEQ ID. NO: 1. For example, probe having SEQ ID NO: 158 has a “g” (guanine) at the complementary position, hence it is operable to selectively hybridize to an allelic variant of the SNP in SEQ ID NO: 1 having a “c” (cytosine) allelic composition, whereas probe having SEQ ID NO: 210 has an “a” (adenine) at the complementary position, hence it is operable to selectively hybridize to an allelic variant of the SNP in SEQ ID NO: 1 having a “t” allelic composition. In some embodiments, both probes may be used to determine what the allelic composition of the SNP in SEQ ID NO: 1. For example, a first probe labeled with a first label may be used to hybridize to one allele of the SNP (of SEQ ID NO. 1 for example having the “c” allelic composition) and a second probe labeled with a second label may be used to hybridize to the other allele of a SNP (of SEQ ID NO: 1, this may be for example the “t” allelic composition. In this example embodiment of SEQ ID NO. 1, if a first probe of SEQ ID NO: 158 and a second probe of SEQ ID NO: 210 are used and only the FAM-MGB signal is detected, the allelic composition of the SNP in SEQ ID NO: 1 is “c.” If however, only the VIC-MGB signal is detected, the SNP allelic composition of the SNP of SEQ ID NO: 1 is “t.”

In some embodiments, a panel of at least 5 and/or at least 10 SNPs selected from the SNPs comprised in SEQ ID NOs: 1-52 may be amplified and the composition of the SNP determined. In some embodiments, all SNPs comprised SEQ ID NOs: 1-52 may be amplified and the allelic composition determined to identify and/or type a strain of S. enterica.

Molecular assays to detect SNPs may include amplification performed by a variety of methods such as but not limited to TaqMan®, SnapShot® and other high throughput screening methods know in the art in light of this specification.

Methods of the disclosure may also comprise determining the allelic compositions (i.e. genotyping and/or SNP detection) by one or more technologies in addition to amplification such as but not limited to sequencing, hybridization, hybridization on bead-based liquid microarray platforms, high throughput screening methods, mass spectrometry, nanostring, microfluidics, chemiluminescence, oligonucleotide ligation, enzyme technologies and combinations thereof. In methods of the disclosure, determining the allelic compositions by sequencing may comprises one or more technologies and/or platforms such as but not limited to genomic sequencing, sequencing targeted regions on CE or semiconductor platforms, multiplex sequencing of all SNP containing regions by CE or semiconductor sequencing and/or by combining with sequencing regions such as, but not limited to, rfb, fliC and fljB regions on the same platform.

Methods of the disclosure may provide one or more advantages listed here. For example, in some embodiments, the methods provide a molecular assay in contrast to traditional immunoassays which may be easier, and/or faster, and/or allow for portable testing options, and/or that may be performed in more accessible settings than traditional serotyping methods.

Some embodiments of the present disclosure provide kits for detection and/or differential detection and/or identification of S enetrica strains. A kit of the disclosure may comprise one or more isolated nucleic acid sequences of the disclosure as set forth herein. Some nucleic acid compositions of the disclosure may comprise primers for amplification of target nucleic acid sequences comprising one or more SNPs that are specific to one or more strains of a S. enterica that may be present in a sample. Some nucleic acid compositions of the disclosure may comprise probes for the detection of target nucleic acid sequences and/or amplified target nucleic acid regions comprising one or more SNPs from a S. enterica strain present in a sample. Probes and primers comprised in kits may be labeled. Kits may additionally comprise one or more components such as, but not limited to: buffers, enzymes, nucleotides, salts, reagents to process and prepare samples (e.g., reagents to isolate nucleic acids), probes, primers, agents to enable detection and control nucleotides. Each component of a kit of the disclosure may be packaged individually or together in various combinations in one or more suitable container means.

While specific advantages have been disclosed hereinabove, it will be understood that various embodiments may include all, some, or none of the previously disclosed advantages. Other technical advantages may become readily apparent to those skilled in the art in light of the teachings of the present disclosure.

These and other features of the present teachings will become more apparent from the detailed description in sections below.

### BRIEF DESCRIPTION OF THE DRAWINGS

One or more embodiments of the present disclosure may be better understood in reference to one or more the drawings below. The skilled artisan will understand that the drawings, described below, are for illustration purposes only. The drawings are not intended to limit the scope of the present teachings in any way.

FIGS. 1A and 1B depict hierarchical clustering of SNP profiles in Salmonella enterica strains, according to one embodiment of the disclosure.

### DETAILED DESCRIPTION

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. Use of “or” means “and/or” unless stated otherwise. The term “and/or” means that the terms before and after can be taken together or separately. For illustration purposes, but not as a limitation, “X and/or Y” can mean “X” or “Y” or “X and Y”.

Whenever a range of values is provided herein, the range is meant to include the starting value and the ending value and any value or value range there between unless otherwise specifically stated. For example, “from 0.2 to 0.5” means 0.2, 0.3, 0.4, 0.5; ranges there between such as 0.2-0.3, 0.3-0.4, 0.2-0.4; increments there between such as 0.25, 0.35, 0.225, 0.335, 0.49; increment ranges there between such as 0.26-0.39; and the like.

The term “cells” refers to the smallest structural unit of an organism that is capable of independent functioning, consisting of one or more nuclei, cytoplasm, and various organelles, all surrounded by a semipermeable cell membrane.

As used herein, the term “contacting” as used herein refers to bringing in contact at least two moieties (reagents, cells, nucleic acids) to bring about a change or a reaction in one or all the moieties. The process of contacting may also comprise “incubating” (contacting for a certain time lengths) and/or incubating at certain temperatures to bring about the change or reaction. In some embodiments “contacting” may also refers to the hybridization between a primer and its substantially complementary region.

The terms “ambient conditions” and “room temperature” are interchangeable and refer to common, prevailing, and uncontrolled atmospheric and weather conditions in a room or place.

As used herein, the term “analyzing” refers to evaluating and comparing the results of a method. In some exemplary embodiments, “analyzing” refers to evaluating and comparing the results of a sample tested to a second sample and/or to a control in a method of the disclosure.

As used herein, “complement” and “complements” are used interchangeably and refer to the ability of a nucleotide, a polynucleotide or two single stranded polynucleotides (for instance, a primer and a target polynucleotide) to base pair with each other, where an adenine on one strand of a polynucleotide will base pair to a thymine or uracil on a strand of a second polynucleotide and a cytosine on one strand of a polynucleotide will base pair to a guanine on a strand of a second polynucleotide. Two polynucleotides are complementary to each other when a nucleotide sequence in one polynucleotide can base pair with a nucleotide sequence in a second polynucleotide. For instance, 5′-ATGC-3′ and 5′-GCAT-3′ are complementary.

As used herein the term “complementary nucleotide sequence” and “complementary sequences” refers to a (second) nucleotide sequence which, by base pairing, is the complement of a first nucleotide sequence. For example, a forward strand with the sequence 5′-ATGGC-3′ would have the complementary nucleotide sequence 3′-TACCG -5′, also termed the “reverse strand.”

The terms “detecting” and “detection” and “determining” are used in a broad sense herein and encompass any technique by which one can determine the absence or presence of something, and/or identify a nucleic acid sequence and/or an exact allelic composition at an SNP locus that may have one or more alleles at that location and/or a protein encoded by a nucleic acid sequence. In some embodiments, detecting comprises quantitating a detectable signal from the nucleic acid, including without limitation, a real-time detection method, such as quantitative PCR (“Q-PCR”), detection of labels. In some embodiments, detecting comprises determining the sequence of an amplification product to determine the sequence of an allele.

As used here, “distinguishing” and “distinguishable” are used interchangeably and refer to differentiating between at least two results from substantially similar or identical reactions, including but not limited to, two different amplification products, two different melting temperatures, two different melt curves, and the like. The results can be from a single reaction, two reactions conducted in parallel, two reactions conducted independently, i.e., separate days, operators, laboratories, and so on. As used herein, “presence” refers to the existence (and therefore to the detection) of a reaction, a product of a method or a process (including but not limited to, an amplification product resulting from an amplification reaction), or to the “presence” and “detection” of an organism such as a pathogenic organism or a particular strain or species of an organism.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “genome” refers to the complete nucleic acid sequence, containing the entire genetic information, of a bacterium, a virus, a plasmid, a gamete, an individual, a population, a species, or a strain of a species.

As used herein, the term “pseudochromosome” refers to the concatenation, in their most likely order, of all available sequence contigs and scaffolds derived from sequencing of a bacterial genome, in which undefined gaps between contigs and scaffolds are represented by unidentified nucleobases.

As used herein, the term “genomic DNA” refers to the chromosomal DNA sequence of a gene or segment of a gene including the DNA sequence of non-coding as well as coding regions. Genomic DNA also refers to DNA isolated directly from cells, chromosomes or plasmid(s) within the genome of an organism, or cloned copies of all or part of such DNA.

### Identification and Selection of SNPs

The present specification relates in some embodiments to identification and selection of several novel single nucleotide polymorphisms (SNPs) that may be used to differentially identify S enterica strains from each other including in some embodiments, to differentiate between closely related strains of Salmonella.

A “single nucleotide polymorphism” or “SNP” refers to a variation in the nucleotide sequence of a polynucleotide that differs from another polynucleotide by a single nucleotide difference. For example, without limitation, exchanging one A for one C, G or T in the entire sequence of polynucleotide constitutes a SNP. It is possible to have more than one SNP in a particular polynucleotide. For example, at one position in a polynucleotide, a C may be exchanged for a T, at another position a G may be exchanged for an A and so on. When referring to SNPs, the polynucleotide is most often DNA.

The present disclosure in some embodiments provides at least 52 SNPs comprised in nucleic acid sequences comprised in SEQ ID. NOs: 1-52 at position 101 in each of these sequences. SEQ ID NOs: 1-52 comprise SNPs of the disclosure at position 101 and are flanked by 100bp of genomic DNA on either side (3′ and 5′ side) of the SNP.

The SNP position in SEQ ID. NOs: 1-52 is indicated by a lowercase letter. The flanking sequences and SNPs are corresponding sequences from a single Salmonella genome (S. Enteritidis, GenBank ID AM933172.1, also described herein as SEQ ID NO. 53). Other Salmonella genomes may differ slightly at other bases within this sequence.

In some embodiments, the disclosure describes compositions comprising isolated nucleic acid sequences having SEQ ID. NOs: 1-52, fragments thereof (including fragments having at least 10 contiguous nucleotides thereof, fragments having at least 20 contiguous nucleotides thereof, fragments having at least 30 contiguous nucleotides thereof, fragments having at least 40 contiguous nucleotides thereof, fragments having at least 50 contiguous nucleotides thereof, and/or fragments having at least 60 contiguous nucleotides thereof), as well as sequences having at least 90% sequence identity, at least 80% sequence identity, and/or at least 70% sequence identity to nucleic acid sequences comprised therein and complementary sequences thereof

In some embodiments, isolated nucleic acid sequences of the disclosure comprising SEQ ID. NOs: 1-52 may have at least 90% sequence identity, at least 80% sequence identity, and/or at least 70% sequence identity to nucleic acid sequences comprised therein.

An isolated SNP-containing nucleic acid molecule may comprise one or more SNP positions disclosed by the present invention with flanking nucleotide sequences on either side of the SNP positions. A flanking sequence can include nucleotide residues that are naturally associated with the SNP site and/or heterologous nucleotide sequences. Although sequences described have 100 bp of flanking nucleic acid sequences, the flanking sequence may be up to about 500, 400, 300, 200, 100, 60, 50, 40, 30, 25, 20, 15, 10, 8, or 4 nucleotides (or any other length in-between) on either side of a SNP position.

In some embodiments, the present disclosure relates to identification of novel SNP loci in Salmonella enterica strains. In order to identify nucleic acid sequences that are conserved in all Salmonella enterica strains, 34 completely sequenced S. enterica serotypes, including 16 presently sequenced strains and 18 publicly available serotypes, were aligned to the S. Enteritidis genome using the open-source whole-genome alignment tool, MUMmer. A list of 34 Salmonella enterica strains are provided in Table 1 attached at the end of the specification. A total of 282 kb of conserved chromosomal sequences were identified. Each of the genomes was then compared to the S. Enteritidis genome (GenBank ID AM933172.1 represented by SEQ ID NO. 53) and 10,101 single nucleotide polymorphisms (SNPs) in these conserved chromosomal regions were identified.

The 10,101 SNPs were screened to identify SNPs specific for different strains. In order to select the most highly discriminative SNPs, the set of 34 genomes was randomly partitioned into two groups 10,000 times, and at each iteration, an SNP with the most highly correlated profile (i.e., most capable of distinguishing between the two random groups) was selected. These were then sorted to obtain the best set of 48 SNPs (shown in Table 2 as SEQ ID NOs: 1-3, 6-13, 15-45, and 47-52. These 48 SNPs identified in the present disclosure vary significantly across the population, but are not associated with any phylogenetic signal. In some embodiments, the 48 SNPs are functional to discriminate between most strains of Salmonella enterica subsp. enterica.

In some embodiments, the disclosure also identifies four additional SNP's operable to differentially identify and discriminate between closely related strains of S. enterica, including between S. Enteritidis and S. Gallinarum; between S. Paratyphi C; and S. Choleraesuis; and between S. Johannesburg and S. Urbana. These four additional SNPs are described in Table 2 as SEQ ID NOs: 4, 5, 14, and 46. These 4 SNPs were manually selected by the present inventors using the selection criteria described above.

Accordingly, the present disclosure identifies a total of 52 unique SNP sequences that are listed in Table 2 as having SEQ ID NOs: 1-52. In some embodiments, the 52 SNPs or subsets selected therefrom are described as an SNP panel. For example an SNP panel of the disclosure may comprise at least 10 SNPs selected from the 52 SNPs identified.

In some embodiments, a SNP panel of the disclosure may be stored in a database such as a database located in a computer readable medium. “Computer readable media” refers to any media which can be read and accessed directly by a computer, and includes, but is not limited to: magnetic storage media such as floppy discs, hard storage medium and magnetic tape; optical storage media such as optical discs or CD-ROM; electrical storage media such as RAM and ROM; and hybrids of these categories, such as magnetic/optical media. By providing such computer readable media, a database comprising SNPs or other data compiled on SNPs may be routinely accessed by a user and used for analysis or designing experiments.

In some embodiments, SNPs of the disclosure may be correlated to Salmonella serotypes and/or strains and an SNP profile database may be created. Some embodiments describe a computer readable medium used to store a SNP profile database of the disclosure.

In some embodiments, SNP profile database and SNP panel databases may be used to design and analyze assays and methods that use a computer system for analysis and identification of Salmonella strains and/or serovars (including differential identification assays and assays correlating SNPs with serotypes). Such methods may also involve data analysis using a “data analysis module” which may include any person or machine, individually or working together, which analyzes the sample and determines the genetic information contained therein. The term may include a person or machine within a laboratory setting.

A “computer system” refers to the hardware means, software means and data storage means used to compile the data of the present invention. The minimum hard ware means of computer-based systems of the invention may comprise a central processing unit (CPU), input means, output means, and data storage means. Desirably, a monitor is provided to visualize structure data. The data storage means may be RAM or other means for accessing computer readable media of the invention. Examples of such systems are microcomputer workstations available from Silicon Graphics Incorporated and Sun Microsystems running Unix based, Linux, Windows NT, XP or IBM OS/2 operating systems.

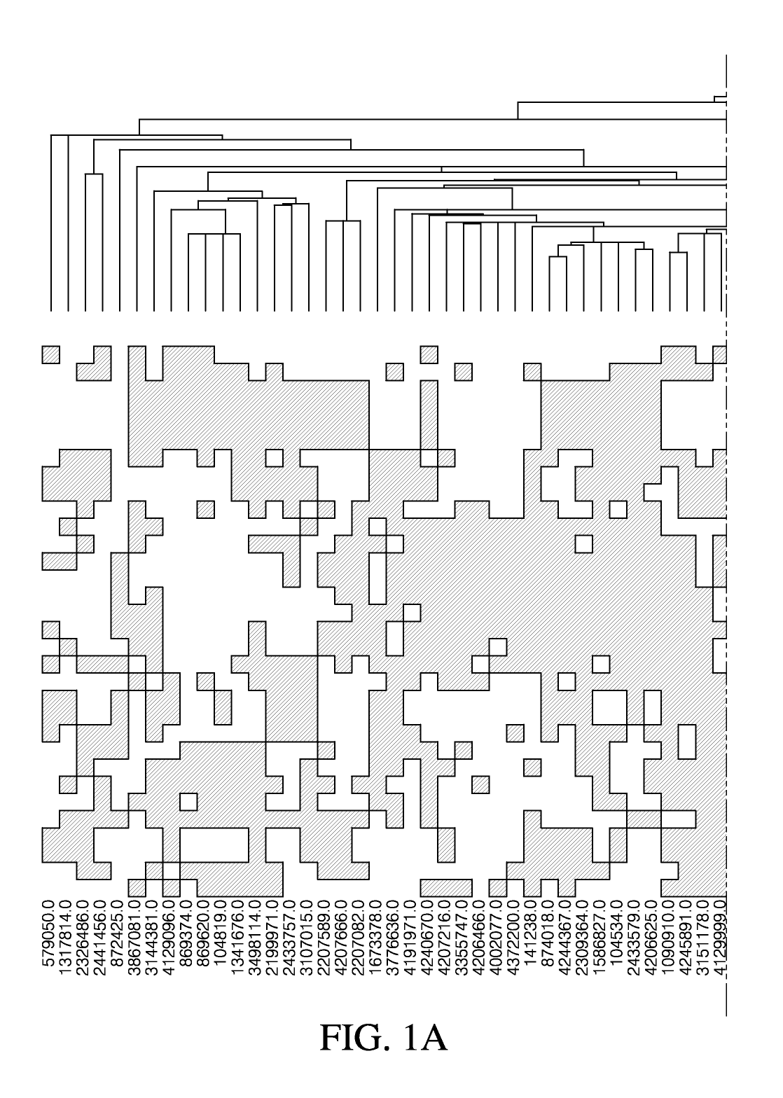

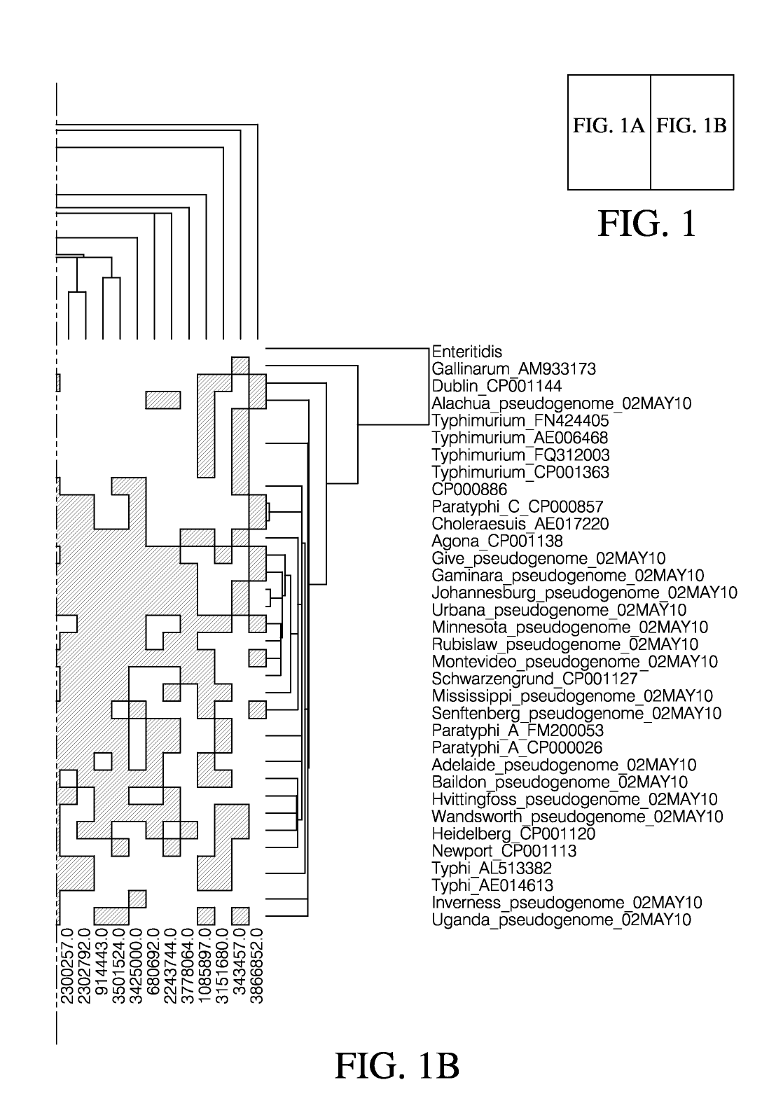

Hierarchical clustering of SNP profiles (depicted in FIGS. 1A and 1B) showed that, in some embodiments, strains of the same serotype have identical profiles. For example, each of four S. Typhimurium strains, each of two S. Typhi strains and each of two S. Paratyphi A strains had identical SNP profiles. The closely related pairs (listed above) differ by two SNPs. Other more unrelated strains differ significantly. For example, the next closely matched pair was the S. Minnesota and the S. Gaminara pair which differed by eight SNPs.

### Validation of SNP Loci

The genetic loci of SNPs identified in the present disclosure were tested and analyzed in several additional strains of S. enterica (including 13 newly available S. enterica genomes—see details below). The 52 SNP loci were found to be present in every strain of S. enterica analyzed by the present inventors.

For example, in order to test the effectiveness of the 52 SNP panel to distinguish new serovars, the genotype at each of the SNP positions was extracted from an additional 13 publicly available draft S. enterica genomes, bringing the total number of genomes analyzed to 47 strains representing 37 serovars. Most new serovars were clearly distinguishable from the set of serovars used to construct the 52 SNP panel. The only exception was serovar 4,5,12:i:-, which could not be differentiated from S. Typhimurium at these SNP positions. These two serovars are known to be very closely related.

Two of the draft genomes are additional isolates of serovars used to construct the panel and comprise the serovars Heidelberg and Schwarzengrund. These have identical profiles to the genomes of completely sequenced Salmonella genomes representing the same serovar. In addition, the two draft S. Kentucky genomes were also identical.

Two of the 13 new serovars tested, including the draft genomes of S. Newport and S. Saintpaul, gave discrepant results. The draft S. Newport genome was found to have a genome profile completely unrelated to the complete Newport genome that used to construct the 52 SNP profile of the present disclosure. Previous work, based on MLST, has demonstrated that Newport isolates can be separated into two distinct evolutionary lineages. Based on the present results it appears that the draft Newport genome represents the other evolutionary lineage. The two draft S. Saintpaul genomes also gave very different profiles. However, as stated at the NCBI genome project pages, “The selected strains are from separate lineages representing genovar groupings: strain SARA23 falls within the main clade for the serovar, and strain SARA29 is an outlier.” Together, the draft S. Newport and S. Saintpaul results indicate that a single serovar may comprise multiple unrelated genetic types, and that these will be reflected in different SNP profiles.

These results further reinforce the stability of the present 52 SNP profile for serovar identification. Accordingly, the present disclosure provides a SNP profile that may be used to establish an interpretable SNP profile for any Salmonella enterica strain using a molecular assay format using the same set-of assay reagents.

In contrast to the present SNPs and SNP profile based assays, another previous SNP based assay targeted a set of five S. enterica serovars. These assays are however limited to be able to identify only the five targeted serovars and do not provide identification of non-targeted serovars (Ben-Darif, JOURNAL OF CLINICAL MICROBIOLOGY, April. 2010, p. 1055-1060 Vol. 48, No. 4).

### Compositions of the Disclosure

The present disclosure, in some embodiments, also provides compositions derived from one or more SNPs identifies here. Accordingly, in some embodiments, oligonucleotides comprising primers operable for amplifying (and identifying) one or more SNPs from the nucleic acid of a S. enterica strain are described. Exemplary primers may comprise isolated nucleic acid sequences comprised in SEQ ID NOs: 54-105 and SEQ ID NOs: 106-157. However, primers of the disclosure are not limited to the sequences and oligonucleotides disclosed in SEQ ID NOs: 54-157, and one of skill in the art, in light of the present teachings will appreciate that additional primers are also disclosed by the present disclosure. For example, in some embodiments, isolated nucleic acid sequences of the disclosure may have at least 90% sequence identity, at least 80% sequence identity, and/or at least 70% sequence identity to nucleic acid sequences comprised in SEQ ID NOs: 54-157. In other examples, any nucleotide having at least 10 nucleotides on one strand and at least 10 nucleotides on a complementary strand of the first strand that flank a SNP of the disclosure to form an amplification product.

In some embodiments, compositions of the disclosure comprise probes operable for identifying one or more SNPs from the nucleic acid of a S. enterica strain are provided. Exemplary probes are described in SEQ ID NOs: 158-209 which correspond to probes for one allele of an SNP, and SEQ ID NOs: 210-261 correspond to example probes that may be used to identify an SNP on the other allele. In the probes described in SEQ ID NOs: 158-261 (see Table 3), the lowercase nucleotide corresponds to the SNP. However, probes of the disclosure are not limited to the sequences and nucleotides disclosed in SEQ ID NOs: 158-261, and one of skill in the art, in light of the present teachings will appreciate that additional probes are also disclosed by the present disclosure. For example, any nucleotide sequence having at least 10 nucleotides, at least 20 nucleotides, at least 30 nucleotides, or at least 40 nucleotides, comprising a SNP may be used as a probe. For example, in non-limiting examples, any nucleotide having at least nucleotides 100-102 of sequences described in SEQ ID NOs: 1-52 and at least 10 additional nucleotides on either the 5′ or the 3′ side of these sequences may be used as a probe of the disclosure. In other non-limiting examples, any nucleotide having at least nucleotides 100-102 of sequences described in SEQ ID NOs: 1-52 and at least 5 additional nucleotides on both the 5′ and the 3′ side of these sequences may be used as a probe of the disclosure. In yet other embodiments, primer sequences of the disclosure may have at least 90% sequence identity, at least 80% sequence identity, and/or at least 70% sequence identity to nucleic acid sequences comprised in SEQ ID NOs: 54-157.

In some embodiments, the disclosure describes compositions comprising isolated nucleic acid sequences having SEQ ID. NOs: 1-52, fragments thereof (including fragments having at least 10 contiguous nucleotides thereof, fragments having at least 15 contiguous nucleotides thereof, fragments having at least 20 contiguous nucleotides thereof, fragments having at least 30 contiguous nucleotides thereof, fragments having at least 40 contiguous nucleotides thereof, fragments having at least 50 contiguous nucleotides thereof, and/or fragments having at least 60 contiguous nucleotides thereof), as well as sequences having at least 90% sequence identity, at least 80% sequence identity, and/or at least 70% sequence identity to nucleic acid sequences comprised therein and complementary sequences thereof. In some embodiments, fragments of the isolated nucleotide sequences derived from SEQ ID NO: 1-SEQ ID NO: 52, as described above, also comprise at least nucleotides located at positions 100-103 of SEQ ID NO: 1-SEQ ID NO: 52, i.e., comprise a SNP of the disclosure. Isolated nucleic acids fragments comprising at least 10 (or more) contiguous nucleotides may be used as primers and/or probes of the disclosure. In some embodiments, isolated nucleic acids fragments comprising at least 10, or at least 15, (or more) contiguous nucleotides and further comprising at least nucleotides located at positions 100-103 of SEQ ID NO: 1-SEQ ID NO: 52, i.e., comprise a SNP of the disclosure may be used as primers and/or probes of the disclosure.

In some embodiments, isolated nucleic acid sequences of the disclosure comprising SEQ ID. NOs: 1-52 may have at least 90% sequence identity, at least 80% sequence identity, and/or at least 70% sequence identity to nucleic acid sequences comprised therein.

Nucleic acids, probes and primers of the disclosure may be further labeled. The term “label” refers to any moiety which can be attached to a molecule and: (i) provides a detectable signal; (ii) interacts with a second label to modify the detectable signal provided by the second label, e.g. FRET; (iii) stabilizes hybridization, i.e. duplex formation; or (iv) provides a capture moiety, i.e. affinity, antibody/antigen, ionic complexation. Labeling can be accomplished using any one of a large number of known techniques employing known labels, linkages, linking groups, reagents, reaction conditions, and analysis and purification methods. Labels include light-emitting compounds which generate a detectable signal by fluorescence, chemiluminescence, or bioluminescence (Kricka, L. in Nonisotopic DNA Probe Techniques (1992), Academic Press, San Diego, pp. 3-28). Another class of labels comprise hybridization-stabilizing moieties which serve to enhance, stabilize, or influence hybridization of duplexes, e.g. intercalators, minor-groove binders, and cross-linking functional groups (Blackburn, G. and Gait, M. Eds. “DNA and RNA structure” in Nucleic Acids in Chemistry and Biology, 2^{nd} Edition, (1996) Oxford University Press, pp. 15-81). Yet another class of labels effect the separation or immobilization of a molecule by specific or non-specific capture, for example biotin, digoxigenin, and other haptens (Andrus, A. “Chemical methods for 5′ non-isotopic labeling of PCR probes and primers” (1995) in PCR 2: A Practical Approach, Oxford University Press, Oxford, pp. 39-54). A label may include but is not limited to a dye, a radioactive isotope, a chemiluminescent label, a fluorescent moiety, a bioluminescent moiety, and/or an enzyme.

As used herein, the terms “polynucleotide”, “oligonucleotide”, and “nucleic acid sequences” are used interchangeably and refer to single-stranded and double-stranded polymers of nucleotide monomers, including without limitation 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺, and the like. A polynucleotide may be composed entirely of deoxyribonucleotides, entirely of ribonucleotides, or chimeric mixtures thereof and can include nucleotide analogs. The nucleotide monomer units may comprise any nucleotide or nucleotide analog. Polynucleotides typically range in size from a few monomeric units, e.g. 5-40 when they are sometimes referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytosine, “G” denotes deoxyguanosine, “T” denotes thymidine, and “U” denotes deoxyuridine, unless otherwise noted.

An “isolated” polynucleotide or oligonucleotide is one that is substantially pure of the materials with which it is associated in its native environment. By substantially free, is meant at least 50%, at least 55%, at least 60%, at least 65%, at advantageously at least 70%, at least 75%, more advantageously at least 80%, at least 85%, even more advantageously at least 90%, at least 91%, at least 92%, at least 93%, at least 94%, at least 95%, at least 96%, at least 97%, most advantageously at least 98%, at least 99%, at least 99.5%, at least 99.9% free of these materials.

An “isolated” nucleic acid molecule is a nucleic acid molecule separate and discrete from the whole organism with which the molecule is found in nature; or a nucleic acid molecule devoid, in whole or part, of sequences normally associated with it in nature; or a sequence, as it exists in nature, but having heterologous sequences in association therewith.

As used herein, the term “nucleotide” or “nt” refers to a base-sugar-phosphate combination. Nucleotides are monomeric units of a nucleic acid molecule (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, 7-deaza-dGTP and 7-deaza-dATP. The term nucleotide as used herein also refers to dideoxyribonucleoside triphosphates (ddNTPs) and their derivatives. Examples of dideoxyribonucleoside triphosphates include, but are not limited to, ddATP, ddCTP, ddGTP, ddITP, and ddTTP.

As used herein, the phrase “nucleic acid molecule” refers to a sequence of contiguous nucleotides (riboNTPs, dNTPs or ddNTPs, or combinations thereof) of any length which can encode a full length polypeptide or a fragment of any length thereof, or which can be non-coding. As used herein, the terms “nucleic acid molecule” and “polynucleotide” can be used interchangeably and include both RNA and DNA.

The terms “identity”, “nucleic acid sequence identity” and “sequence identity” are used interchangeably and refer to the percentage of pair-wise identical residues—following homology alignment of a sequence of a polynucleotide with a sequence in question—with respect to the number of residues in the longer of these two sequences. The term “identity” as known in the art refers to a relationship between the sequences of two or more polypeptide molecules or two or more nucleic acid molecules, as determined by comparing the sequences. In the art, “identity” also means the degree of sequence relatedness between nucleic acid molecules or polypeptides, as the case may be, as determined by the match between strings of two or more nucleotide or two or more amino acid sequences. “Identity” measures the percent of identical matches between the smaller of two or more sequences with gap alignments (if any) addressed by a particular mathematical model or computer program (i.e., “algorithms”).

The term “percent (%) nucleic acid sequence identity” with respect to a nucleic acid sequence refers to the percentage of nucleotides in a first sequence that are identical with the nucleotides in a second nucleic acid sequence of interest, after aligning the sequences and introducing gaps, if necessary, to achieve the maximum percent sequence identity. Alignment for purposes of determining percent nucleic acid sequence identity can be achieved in various ways that are known to one of skill in the art, for instance, using publicly available computer software such as BLAST, BLAST-2, ALIGN or Megalign (DNASTAR) software.

Percent nucleic acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, MD. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

### Methods Using a SNP Panel of the Disclosure

The present disclosure, in some embodiments, describes methods for identifying and/or distinguishing and/or differentially detecting a Salmonella enterica strain and serovars from a sample and the method comprises detecting the presence or absence of one or more SNPs identified herein.

As used herein the term “sample” may refer to a starting material suspected of harboring a particular strain or serovar of Salmonella enterica. A “contaminated sample” refers to a sample harboring a Salmonella enterica microbe thereby comprising nucleic acid material from the microbe. Examples of samples include, but are not limited to, food samples (including but not limited to samples from food intended for human or animal consumption such as processed foods, raw food material, produce (e.g., fruit and vegetables), legumes, meats (from livestock animals and/or game animals), fish, sea food, nuts, beverages, drinks, fermentation broths, and/or a selectively enriched food matrix comprising any of the above listed foods), water samples, environmental samples (e.g., soil samples, dirt samples, garbage samples, sewage samples, industrial effluent samples, air samples, or water samples from a variety of water bodies such as lakes, rivers, ponds etc.,), air samples (from the environment or from a room or a building), forensic samples, agricultural samples, pharmaceutical samples, biopharmaceutical samples, samples from food processing and manufacturing surfaces, and/or biological samples. A “biological sample” refers to a sample obtained from mammals: such as a human, a cow, a pig, a livestock animal, a rabbit, a game animal, and/or a member of the family Muridae (a murine animal such as rat or mouse); and other animals and birds such as a chicken, a turkey, a fish, a crab, a crustacean. A biological sample may include blood, urine, feces, or other materials from a human or a livestock animal. A biological sample can be, for instance, in the form of a single cell, in the form of a tissue, or in the form of a fluid.

Prior to detecting the presence of one or more SNPs or a SNP panel in the nucleic acid contained in a sample, a sample may be prepared and/or processed. As used herein “preparing” or “preparing a sample” or “processing” or processing a sample” refers to one or more of the following steps to achieve extraction and separation of a nucleic acid from a sample: (1) bacterial enrichment, (2) separation of bacterial cells from the sample, (3) cell lysis, and (4) nucleic acid extraction and/or purification (e.g., DNA extraction, total DNA extraction, genomic DNA extraction, RNA extraction). Nucleic acid extracted may then be analyzed for presence of one or more SNP.

A method of identifying a strain or serovar of Salmonella enterica in a sample comprises: testing a nucleic acid isolated from the sample to determine an allele corresponding to a single nucleotide polymorphisms (SNP) for at least ten SNPs; and determining the allelic composition for the at least ten SNPs selected from the panel of SNPs, wherein the presence of certain alleles identifies the strain or serovar of Salmonella enterica.

In some embodiments, determining an allele corresponding to a single nucleotide polymorphism (SNP) for at least ten SNPs comprises determining the allelic composition of at least 10 SNP's from a panel of fifty two SNPs comprised at position 101 in nucleic acid sequences described in SEQ ID NO: 1 - SEQ ID NO: 52 or complementary sequences thereof.

In some embodiments, a method of the disclosure further comprises the steps of: creating a SNP profile of the sample nucleic acids comprising the allelic composition of each SNPs for the at least ten SNPs selected from a panel of SNPs (such as ,for example, a panel of fifty two SNPs identified herein or larger or smaller panels of SNP's); comparing the SNP profile of the sample nucleic acids with a database of Salmonella enterica strains SNP profiles; and determining the strain or serovar of the Salmonella enterica comprised in the sample, wherein the presence of certain alleles identifies the strain or serovar of Salmonella enterica.

A method of identifying a strain or serovar of Salmonella enterica in a sample can comprise: determining an allele corresponding to a single nucleotide polymorphisms (SNP) for at least ten SNPs from nucleic acids isolated from the sample; and determining the allelic composition for the at least ten SNPs selected from a panel of SNPs, wherein the presence of certain alleles identifies the strain or serovar of Salmonella enterica. The method can further comprising the steps of: creating a SNP profile of the sample nucleic acids comprising the allelic composition of each SNPs for the at least ten SNPs selected from the panel of SNPs; comparing the SNP profile of the sample nucleic acids with a database of Salmonella enterica strains SNP profiles; and determining the strain or serovar of the Salmonella enterica comprised in the sample, wherein the presence of certain alleles identifies the strain or serovar of Salmonella enterica. In some exemplary embodiments, the panel of SNPs comprises fifty two SNPs comprised at position 101 in nucleic acid sequences described in SEQ ID NO: 1-SEQ ID NO: 52 or complementary sequences thereof.

In some embodiments of the methods, determining the allelic composition comprises testing the nucleic acid isolated from the sample to determine an allele corresponding to a single nucleotide polymorphisms (SNP) wherein the testing comprises the steps of: a) identifying at least a first target nucleic acid sequence comprising a first SNP from the at least 10 SNPs selected from the panel of fifty two SNPs comprised at position 101 in nucleic acid sequences described in SEQ ID NO: 1-SEQ ID NO: 52 or complementary sequences thereof; b) hybridizing at least a first pair of polynucleotide primers to the first target nucleic acid sequence comprising a first SNP; c) amplifying the first target nucleic acid sequence to form a first amplified target nucleic acid sequence product comprising the first SNP; d) determining the allelic composition of the first SNP from the first amplified target nucleic acid sequence product comprising the first SNP; e) repeating steps a)-d) using a different set of primer pairs, each primer pair operable to hybridize to and amplify a target nucleic acid sequence comprising another SNP and determining the allelic composition of each SNP until the allelic composition of the at least 10 SNPs are determined.

In some embodiments, a method of the disclosure may comprise detecting the allelic composition of a panel of at least 10 SNPs to identify and/or differentially detect a strain or a serovar of S. enterica from a sample. In some embodiments, a method may comprise detecting allelic composition of all the 52 SNPs to identify and/or differentially detect a strain or a serovar of S. enterica.

Determining the allelic compositions and/or genotyping and/or SNP detection may comprises one or more technologies such as but not limited to sequencing (also see the next sentence), amplification, hybridization, bead-based liquid microarray platforms, high throughput screening methods, mass spectrometry, nanostring, microfluidics, chemiluminescence, oligonucleotide ligation, enzyme technologies and combinations thereof Determining the allelic compositions and/or genotyping and/or SNP detection by sequencing may comprises one or more technologies and/or platforms such as but not limited to genomic sequencing, sequencing targeted regions on CE or semiconductor platforms, multiplex sequencing of all SNP containing regions by CE or semiconductor sequencing and/or by combining with sequencing regions such as but not limited to rfb, fliC and fljB regions on the same platform. Molecular assays to detect SNPs may include amplification performed by a variety of methods such as but not limited to TaqMan®, SnapShot® and other high throughput screening methods know in the art in light of this specification.

Some methods for identifying and/or detecting S. enterica strains in a sample may comprise using an isolated nucleotide sequence composition of the disclosure for detection. Exemplary compositions of the disclosure used for detection methods may comprise, but are not limited to, SEQ ID NO: 54-157, and/or SEQ ID NO:158-261, fragments thereof, at least 10 contiguous nucleotide sequences thereof, complements thereof, isolated nucleic acid sequence comprising at least 90% nucleic acid sequence identity to the sequences set forth above and/or labeled derivatives thereof.

In some embodiments, methods of the disclosure may comprise: isolating a nucleic acid from a sample suspected of having a S. enterica strain and/or a sample from which one desires to detect and/or identify a specific S. enterica strain; amplifying one or more SNP comprising nucleic acid sequences (nucleotides) from the nucleic acid from the sample to form an amplification product; and detecting and determining the allelic composition of an amplification product comprising an SNP, correlating the allelic composition of the SNP in the nucleic acid of the sample to known S. enterica strain/serovar allelic compositions. Amplification reactions may be multiplexed to detect a panel of SNPs. In some embodiments, a panel of at least 10 SNPs selected from the SNPs comprised in SEQ ID NOs: 1-52 may be amplified and detected. In some embodiments, all SNPs comprised SEQ ID NOs: 1-52 may be amplified and detected to identify and/or type a strain of S. enterica.

In some embodiments, a pair of primers used for amplification may comprise the nucleic acid sequence of SEQ ID NO: 54-105, and/or SEQ ID NO: 106-157, fragments thereof, at least 10 contiguous nucleotide sequences thereof, complements thereof, isolated nucleic acid sequence comprising at least 90% nucleic acid sequence identity to the sequences set forth above and/or labeled derivatives thereof. For example, as shown in Table 3, a primer pair shown as reverse and forward primers may be used to amplify the corresponding SNP in the same row. Thus for example, a primer pair may comprise a first primer and a second primer, the first primer having a SEQ ID NO: 54 may be used as a forward primer, the second primer having a nucleic acid of SEQ ID NO: 106 may be used as a reverse primer may be used to amplify a SNP comprised in SEQ ID NO 1. Primers may be labeled and nucleic acid amplification products may be detected and/or identified by a variety of methods known in the art including but not limited to size analysis of an amplified product; sequencing an amplified product; hybridization with a probe; 5′ nuclease digestion; single-stranded conformation polymorphism; allele specific hybridization; primer specific extension; and oligonucleotide ligation assay.

The term “primer” refers to a polynucleotide and analogs thereof that are capable of selectively hybridizing to a target nucleic acid or a “template,” a target region flanking sequence or to a corresponding primer-binding site of an amplification product; and allows detection of a double-stranded nucleic acid formed by hybridization or the synthesis of a sequence complementary to the corresponding polynucleotide template, flanking sequence or amplification product from the primer's 3′ end. Typically a primer can be between about 10 to 100 nucleotides in length and can provide a point of initiation for template-directed synthesis of a polynucleotide complementary to the template, which can take place, in the presence of appropriate enzyme(s), cofactors, substrates such as nucleotides and the like.

As used herein, the term “amplification primer” refers to an oligonucleotide, capable of annealing to an RNA or DNA region adjacent a target nucleic acid sequence, and serving as an initiation primer for nucleic acid synthesis under suitable conditions well known in the art. Typically, a PCR reaction employs a pair of amplification primers including an “upstream” or “forward” primer and a “downstream” or “reverse” primer, which delimit a region of the RNA or DNA to be amplified.

As used herein, the term “primer-binding site” refers to a region of a polynucleotide sequence, typically a sequence flanking a target region and/or an amplicon that can serve directly, or by virtue of its complement, as the template upon which a primer can anneal for any suitable primer extension reaction known in the art, for example, but not limited to, PCR. It will be appreciated by those of skill in the art that when two primer-binding sites are present on a single polynucleotide, the orientation of the two primer-binding sites is generally different. For example, one primer of a primer pair is complementary to and can hybridize with the first primer-binding site, while the corresponding primer of the primer pair is designed to hybridize with the complement of the second primer-binding site. Stated another way, in some embodiments the first primer-binding site can be in a sense orientation, and the second primer-binding site can be in an antisense orientation. A primer-binding site of an amplicon may, but need not comprise the same sequence as or at least some of the sequence of the target flanking sequence or its complement.

The terms “amplifying” and “amplification” are used in a broad sense and refer to any technique by which a target region, an amplicon, or at least part of an amplicon, is reproduced or copied (including the synthesis of a complementary strand), typically in a template-dependent manner, including a broad range of techniques for amplifying nucleic acid sequences, either linearly or exponentially. Some non-limiting examples of amplification techniques include primer extension, including the polymerase chain reaction (PCR), reverse transcription polymerase chain reaction (RT-PCR), asynchronous PCR (A-PCR), and asymmetric PCR (AM-PCR), strand displacement amplification (SDA), multiple displacement amplification (MDA), nucleic acid strand-based amplification (NASBA), rolling circle amplification (RCA), transcription-mediated amplification (TMA), and the like, including multiplex versions, and combinations thereof Descriptions of certain amplification techniques can be found in, among other places, Molecular Cloning, A Laboratory Manual, Cold Spring Harbor Press, 3d ed., 2001 (hereinafter “Sambrook and Russell”); Sambrook et al.; Ausubel et al.; PCR Primer: A Laboratory Manual, Diffenbach, Ed., Cold Spring Harbor Press (1995); Msuih et al., J. Clin. Micro. 34:501-07 (1996); McPherson; Rapley; U.S. Pat. Nos. 6,027,998 and 6,511,810; PCT Publication Nos. WO 97/31256 and WO 01/92579; Ehrlich et al., Science 252:1643-50 (1991); Favis et al., Nature Biotechnology 18:561-64 (2000); Protocols & Applications Guide, rev. 9/04, Promega, Madison, Wis.; and Rabenau et al., Infection 28:97-102 (2000).

The terms “amplicon,” “amplification product” and “amplified sequence” are used interchangeably herein and refer to a broad range of techniques for increasing polynucleotide sequences, either linearly or exponentially and can be the product of an amplification reaction. An amplicon can be double-stranded or single-stranded, and can include the separated component strands obtained by denaturing a double-stranded amplification product. In certain embodiments, the amplicon of one amplification cycle can serve as a template in a subsequent amplification cycle. Exemplary amplification techniques include, but are not limited to, PCR or any other method employing a primer extension step. Other non-limiting examples of amplification include, but are not limited to, ligase detection reaction (LDR) and ligase chain reaction (LCR). Amplification methods can comprise thermal-cycling or can be performed isothermally. In various embodiments, the term “amplification product” and “amplified sequence” includes products from any number of cycles of amplification reactions.

As used herein, the “polymerase chain reaction” or PCR comprises amplification of a nucleic acid consisting of an initial denaturation step which separates the strands of a double stranded nucleic acid sample, followed by repetition of (i) an annealing step, which allows amplification primers to anneal specifically to positions flanking a target sequence; (ii) an extension step which extends the primers in a 5′ to 3′ direction thereby forming an amplicon polynucleotide complementary to the target sequence, and (iii) a denaturation step which causes the separation of the amplicon from the target sequence (Mullis et al., EDS, The Polymerase Chain Reaction, BirkHauser, Boston, Mass. (1994)). Each of the above steps may be conducted at a different temperature, preferably using an automated thermocycler (Applied Biosystems LLC, a division of Life Technologies Corporation, Foster City, Calif.). If desired, RNA samples can be converted to DNA/RNA heteroduplexes or to duplex cDNA by methods known to one of skill in the art. PCR methods may also include reverse transcriptase-PCR and other reactions that follow principles of PCR.

An “amplified polynucleotide” may be a SNP-containing nucleic acid molecule whose amount has been increased at least two fold by any nucleic acid amplification method performed in vitro as compared to its starting amount in a test sample. In some embodiments, an amplified polynucleotide may be the result of at least ten fold, fifty fold, one hundred fold, one thousand fold, or even ten thousand fold increase as compared to its starting amount in a test sample. In a typical PCR amplification, a polynucleotide of interest is often amplified at least fifty thousand fold in amount over the unamplified genomic DNA, but the precise amount of amplification needed for an assay depends on the sensitivity of the subsequent detection method used.

Generally, an amplified polynucleotide is at least about 16 nucleotides in length. More typically, an amplified polynucleotide is at least about 20 nucleotides in length. In some embodiments, an amplified polynucleotide is at least about 30 nucleotides in length and may be at least about 32, 40, 45, 50, or 60 nucleotides in length. In some embodiments, an amplified polynucleotide is at least about 100, 200, or 300 nucleotides in length. While the total length of an amplified polynucleotide may be as long as an exon, an intron or the entire gene where an SNP of interest resides, an amplified product is typically no greater than about 1,000 nucleotides in length (although certain amplification methods may generate amplified products greater than 1000 nucleotides in length). More preferably, an amplified polynucleotide is not greater than about 600 nucleotides in length. It is understood that irrespective of the length of an amplified polynucleotide, an SNP of interest may be located anywhere along its sequence.

In some embodiments, methods of the disclosure may comprise: isolating a nucleic acid from a sample suspected of having a S. enterica strain and/or a sample from which one desires to detect and/or identify a specific S. enterica strain; hybridizing one or more SNP comprising regions of the nucleic acid from the sample using one or more probes, each probe designed to bind specifically to a region comprising an SNP; and detecting the hybridized probe-nucleic acid complex. Probes used may be labeled to enable detection. Multiplex hybrid detection maybe enable by using differentially labeled probes. Other detection methods may comprise size analysis of the hybridized product; sequencing an amplified product; hybridization with a probe; 5′ nuclease digestion; single-stranded conformation polymorphism; and/or allele specific hybridization. These and other methods of detecting probes and identifying nucleic acids bound to probes are known to one of skill in the art in light of this disclosure and may be used.

In some embodiments, probes used for hybridization and/or for detection of amplified products comprising a SNP may comprise the nucleic acid sequence of SEQ ID NO: 158-209, and/or SEQ ID NO: 210-261 and/or labeled derivatives thereof. For example, as shown in Table 3, probes 1 labeled with FAM-MGB and/or probes 2 labeled with VIC-MGB may be used to identify the allele of the corresponding SNP in the same row. Thus for example, a probe having SEQ ID NO: 158 may be used to hybridize to one allele of a SNP comprised in SEQ ID NO: 1, a probe having SEQ ID NO: 210 may be used to hybridize to the other allele of the SNP comprised in SEQ ID NO: 1. In some embodiments, both probes may be used. For example a first probe labeled with a first label may be used to hybridize to one allele of a SNP and a second probe labeled with a second label may be used to hybridize to the other allele of a SNP. In an example embodiment, a first probe may have SEQ ID NO: 158 and a second probe may have SEQ ID NO: 210 and this set of probes may be used to hybridize to and detect a SNP comprised in SEQ ID NO 1.

The terms “reporter probe” and “probe” are used interchangeably and refer to a detectable sequence of nucleotides or a detectable sequence of nucleotide analogs operable to specifically anneal with a corresponding amplicon, such as but not limited to, a target nucleic acid sequence and/or a PCR product and is further operable to be detected or identified. Reporter probes or probes may be detectable by a variety of methods, including but not limited to, detecting color, detecting radiation, fluorescence, luminescence, emitted wavelengths. In some embodiments, detecting a change in intensity, a change in radiation, a change in an emitted wavelength, a change in fluorescence, a change in luminescence, or a change in color or intensity of color may be used to identify and/or quantify a corresponding amplicon or a target polynucleotide. In one exemplary embodiment, by indirectly detecting an amplicon from a sample or processed sample, one can determine that a microorganism having a corresponding target sequence is present in a sample. Most reporter probes can be categorized based on their mode of action, for example but not limited to: nuclease probes, including without limitation TaqMan° probes; extension probes including without limitation scorpion primers, Lux™ primers, Amplifluors, and the like; and hybridization probes including without limitation molecular beacons, Eclipse probes, light-up probes, pairs of singly-labeled reporter probes, hybridization probe pairs, and the like. In certain embodiments, reporter probes may comprise an amide bond, an LNA, a universal base, and/or combinations thereof, and may include stem-loop and/or stem-less reporter probe configurations. Certain reporter probes may be singly-labeled, while other reporter probes are doubly-labeled. Dual probe systems that comprise FRET between adjacently hybridized probes are within the intended scope of the term reporter probe. In certain embodiments, a reporter probe may comprise a fluorescent reporter group and a quencher (including without limitation dark quenchers and fluorescent quenchers). Some non-limiting examples of reporter probes include TaqMan® probes; Scorpion probes (also referred to as scorpion primers); Lux™ primers; FRET primers; Eclipse probes; molecular beacons, including but not limited to FRET-based molecular beacons, multicolor molecular beacons, aptamer beacons, PNA beacons, and antibody beacons; labeled PNA clamps, labeled PNA openers, labeled LNA probes, and probes comprising nanocrystals, metallic nanoparticles and similar hybrid probes (see, e.g., Dubertret et al., Nature Biotech., 19:365-70, 2001; Zelphati et al., BioTechniques 28:304-15, 2000). In certain embodiments, reporter probes may further comprise minor groove binders including but not limited to TaqMan® MGB probes and TaqMan® MGB-NFQ probes (both from Applied Biosystems). In certain embodiments, reporter probe detection may comprise fluorescence polarization detection (see, e.g., Simeonov and Nikiforov, Nucl. Acids Res. 30:E91, 2002).

“Hybridization” refers to a process in which single-stranded nucleic acids with complementary or near-complementary base sequences interact to form hydrogen-bonded complexes called hybrids. Hybridization reactions are sensitive and selective. In vitro, the specificity of hybridization (i.e., stringency) is controlled by factors such as the concentrations of salt or formamide in prehybridization and hybridization solutions and by the hybridization temperature. In some embodiments, stringency may be increased by reducing the concentration of salt, increasing the concentration of formamide, and/or by raising the hybridization temperature. For example, high stringency conditions could occur at about 50% formamide at 37° C. to 42° C. Reduced stringency conditions could occur at about 35% to 25% formamide at 30° C. to 35° C. Some examples of stringency conditions for hybridization are also described in Sambrook, J., 1989, Molecular Cloning A Laboratory Manual, Cold Spring Harbor Laboratory Press, Cold Spring Harbor, N.Y. Generally, the temperature for hybridization is about 5-10° C. less than the melting temperature (Tm) of a hybrid nucleic acid.

As used herein, the term “homology” refers to a degree of complementarity at the nucleic acid level that can be determined by known methods, e.g. computer-assisted sequence comparisons (Basic local alignment search tool, S. F. Altschul et al., J. Mol. Biol. 215 (1990), 403 410). The term “homology” known to the skilled person describes the degree to which two or more nucleic acid molecules are related, this being determined by the concordance between the sequences. The percentage of “homology” is obtained from the percentage of identical regions in two or more sequences, taking into account gaps or other sequence peculiarities. The homology of nucleic acid molecules which are related to one another can be determined with the aid of known methods. As a rule, special computer programs with algorithms which take account of the particular requirements are employed. There can be partial homology or complete homology (i.e., identity). A partially complementary sequence that at least partially inhibits a completely complementary sequence from hybridizing to a target nucleic acid is referred to using the functional term “substantially homologous.”

The term “selectively hybridize” and variations thereof means that under appropriate stringency conditions, a given sequence (for example, but not limited to, a primer) anneals with a second sequence comprising a complementary string of nucleotides (for example but not limited to a target flanking sequence or a primer-binding site of an amplicon), but does not anneal to undesired sequences, such as non-target nucleic acids or other primers. Typically, as the reaction temperature increases toward the melting temperature of a particular double-stranded sequence, the relative amount of selective hybridization generally increases and mis-priming generally decreases. In this specification, a statement that one sequence hybridizes or selectively hybridizes with another sequence encompasses situations where the entirety of both of the sequences hybridize to one another and situations where only a portion of one or both of the sequences hybridizes to the entire other sequence or to a portion of the other sequence.

Methods of the disclosure as described above may be adapted to a number of assay formats may be designed for genotyping using the identified SNPs, and these may include assays such as but not limited to: a 5′ nuclease assays (e.g., a TaqMan assay, an embodiment of which is described in the section titled Examples); a high resolution melting (HRM) analysis; a molecular beacon assay; a microarray hybridization; a primer extension assay (e.g., SNaPshot); and a oligonucleotide ligation assay (OLA; e.g., SNPplex).

### Applications of the SNP Panel

The panel of SNPs described here may be developed into assays as described above, and used in a variety of applications to characterize the serotype or subtype of a Salmonella strain that has been isolated. These applications may include: routine Salmonella strain typing; food testing, environmental testing, clinical microbiology testing, veterinary microbiology testing, and/or outbreak source tracking.

A variety of samples may be tested for identifying Salmonella serovars and strains for these applications and may include samples such as but not limited to food samples, environmental samples, clinical samples, industrial samples, laboratory samples, air samples, water samples.

### Kits

Also provided are kits comprising SNP detection reagents. Kits of the present disclosure may be employed for detection and/or differential detection and/or identification of S enetrica strains. A kit of the disclosure may comprise one or more isolated nucleic acid sequences of the disclosure as set forth herein. Some nucleic acid compositions of the disclosure may comprise primers for amplification of target nucleic acid sequences comprising one or more SNPs that are specific to one or more strains of a S. enterica that may be present in a sample. Some nucleic acid compositions of the disclosure may comprise probes for the detection of target nucleic acid sequences and/or amplified target nucleic acid regions comprising one or more SNPs from a S. enterica strain present in a sample. Probes and primers comprised in kits may be labeled.

Kits may additionally comprise one or more components such as, but not limited to: buffers, enzymes, nucleotides, salts, reagents to process and prepare samples (e.g., reagents to isolate nucleic acids), probes, primers, agents to enable detection and control nucleotides and reagents and/or platforms for nucleic acid sequencing to identify and/or determine SNPs.

A kit may further comprise reagents for downstream processing of an isolated nucleic acid and may include without limitation at least one RNase inhibitor; at least one cDNA construction reagents (such as reverse transcriptase); one or more reagents for amplification of RNA, one or more reagents for amplification of DNA including primers, reagents for purification of DNA, probes for detection of specific nucleic acids.

Each component of a kit of the disclosure may be packaged individually or together in various combinations in one or more suitable container means. A container means may generally comprise at least one vial, test tube, flask, bottle, syringe or other container means, into which a component may be placed, and preferably, suitably aliquoted. Where there are more than one component in a kit they may be packaged together if suitable or the kit will generally contain a second, third or other additional container into which the additional components may be separately placed. However, in some embodiments, certain combinations of components may be packaged together comprised in one container means. A kit can also include a means for containing the DNA/RNA, and any other reagent containers in close confinement for commercial sale. Such containers may include injection or blow-molded plastic containers into which the desired vials are retained.

Some components of a kit are provided in one and/or more liquid solutions. Liquid solution may be non-aqueous solution, an aqueous solution, and may be a sterile solution.

Components of the kit may also be provided as dried powder(s). When reagents and/or components are provided as a dry powder, the powder can be reconstituted by the addition of a suitable solvent. It is envisioned that a suitable solvent may also be provided in another container means. Kits may also comprise a container means for containing a sterile, pharmaceutically acceptable buffer and/or other diluent.

A kit of the disclosure may also include instructions for employing the kit components and may also have instructions for the use of any other reagent not included in the kit. Instructions can include variations that can be implemented.

The section headings used herein are for organizational purposes only and are not to be construed as limiting the subject matter described in any way. All literature and similar materials cited in this application including, but not limited to, patents, patent applications, articles, books, treatises, and internet web pages, regardless of the format of such literature and similar materials, are expressly incorporated by reference in their entirety for any purpose. In the event that one or more of the incorporated literature and similar materials defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents that will be appreciated by those of skill in the art in light of these teachings.

### EXAMPLES

Aspects of the present teachings can be further understood in light of the following examples, which should not be construed as limiting the scope of the present teachings in any way.

### Example 1

### TaqMan SNP Genotyping Assays

A number of assay formats may be used for genotyping SNPs described herein. In one example embodiment, a TaqMan SNP genotyping assay was performed. Using proprietary Taqpipe software, two-dye SNP genotyping assays were designed. These assays were successfully validated in the laboratory by running the assays in microtiter plate format against a panel of six strains, the genomes of which were used in identification of the SNP panel. Results were as expected.

In some embodiments, one or more genotyping assays of the disclosure may be run using the 52 SNP panel described herein on a collection of commonly encountered Salmonella serotypes, and a validated database of SNP profiles observed for each of these serotypes may be established. While analyzing a sample to test for the presence of one or more Salmonella serotypes, 52 SNP TaqMan assays may be performed and the assays may be loaded into a multiwall format plate (such as an OpenArray plate) so that a number of strains may be analyzed, in parallel, against all of the 52 SNP assays.

Prior to serotype testing, the strains to be tested will typically be cultured, and genomic DNA isolated. These DNA templates would be added to a multiwell format (such as an OpenArray slide), and run through a DNA amplification method (such as a thermal cycling protocol) and an endpoint fluorescence protocol. A software tool may be used to automate the allele calling for each SNP assay, and thereby an entire 52-SNP profile may be obtained. This 52-SNP profile may be searched against the database of validated Salmonella serotypes in order to identify high confidence matches that would identify the serotype of the strain being tested.

### Example 2

### Additional SNP Identification and increasing SNP Panel Repertoire

As described earlier, the present inventors manually selected distinguishing SNPs for very closely related strains. For example 4 SNPs were manually selected for distinguishing between S. Enteritidis and S. Gallinarum; between S. Paratyphi C; and S. Choleraesuis; and between S. Johannesburg and S. Urbana. Additional SNPs are contemplated to be further identified by manual selection and/or other selection criteria for other closely related strains, which is expected to incrementally add new SNP markers to a SNP panel for serovar/strain identification assays.

### Example 3

### Association of SNPs with Serotype Determining loci

The present inventors also contemplate associating one or more identified SNPs with a serotype determination loci. For example, a SNP may be located with a serotype determining locus such as with a flagellar genes, or O-antigen biosynthetic genes. For this, the profile of each known reference serotype will be correlated with each SNP profile. In some embodiments, this may comprise typing several (e.g., thousands) of Salmonella strains from large reference collections in order to establish a validated database of serotype profiles and correlating them with a SNP profiles database to allow serotype determination.

However, in some embodiments, strains that are closely related by SNP profile do not necessarily have similar serotypes: for example, Urbana (30:a:e,n,x) and Johannesburg (1,40:b:e,n,x) are antigenically distinct, but very similar by core SNP profile. This may be due to factors such as antigenic switching by recombination at the antigen determining loci.

TABLE 1
S. enterica Strains employed

| | | GenBank | | Used for SNP | Used for SNP |
|---|---|---|---|---|---|
| Serovar | Strain | accession | Sequence source | identification | validation |
| 4-5-12:i:- | CVM23701 | ABAO00000000 | GenBank | No | yes |
| Adelaide | A4-669 | AFCI00000000 | Internal sequencing | yes | yes |
| Agona | SL483 | CP001138 | GenBank | yes | yes |
| Alachua | R6-377 | AFCJ00000000 | Internal sequencing | yes | yes |
| Baildon | R6-199 | AFCK00000000 | Internal sequencing | yes | yes |
| Choleraesuis | SC-B67 | AE017220 | GenBank | yes | yes |
| Dublin | CT_02021853 | CP001144 | GenBank | yes | yes |
| Enteritidis | P125109 | AM933172 | GenBank | yes (reference) | yes |
| Gallinarum | 287/91 | AM933173 | GenBank | yes | yes |
| Gaminara | A4-567 | AFCL00000000 | Internal sequencing | yes | yes |
| Give | S5-487 | AFCM00000000 | Internal sequencing | yes | yes |
| Hadar | RI_05P066 | ABFG00000000 | GenBank | No | yes |
| Heidelberg | SL476 | CP001120 | GenBank | yes | yes |
| Heidelberg | SL486 | ABEL00000000 | GenBank | No | yes |
| Hvittingfoss | A4-620 | AFCN00000000 | Internal sequencing | yes | yes |
| Inverness | R8-3668 | AFCO00000000 | Internal sequencing | yes | yes |
| Javiana | GA_MM04042433 | ABEH00000000 | GenBank | No | yes |
| Johannesburg | S5-703 | AFCP00000000 | Internal sequencing | yes | yes |
| Kentucky | CDC191 | ABEI00000000 | GenBank | No | yes |
| Kentucky | CVM29188 | ABAK00000000 | GenBank | No | yes |
| Minnesota | A4-603 | AFCQ00000000 | Internal sequencing | yes | yes |
| Mississippi | A4-633 | AFCR00000000 | Internal sequencing | yes | yes |
| Montevideo | S5-403 | AFCS00000000 | Internal sequencing | yes | yes |
| Newport | SL254 | CP001113 | GenBank | yes | yes |
| Newport | SL317 | ABEW00000000 | GenBank | No | yes |
| Paratyphi A | ATCC 9150 | CP000026 | GenBank | yes | yes |
| Paratyphi A | AKU_12601 | FM200053 | GenBank | yes | yes |
| Paratyphi B | SPB7 | CP000886 | GenBank | yes | yes |
| Paratyphi C | RKS4594 | CP000857 | GenBank | yes | yes |
| Rubislaw | A4-653 | AFCT00000000 | Internal sequencing | yes | yes |
| Saintpaul | SARA23 | ABAM00000000 | GenBank | No | yes |
| Saintpaul | SARA29 | ABAN00000000 | GenBank | No | yes |
| Schwarzengrund | CVM19633 | CP001127 | GenBank | yes | yes |
| Schwarzengrund | SL480 | ABEJ00000000 | GenBank | No | yes |
| Senftenberg | A4-543 | AFCU00000000 | Internal sequencing | yes | yes |
| Tennessee | CDC07-0191 | ACBF00000000 | GenBank | No | yes |
| Typhi | Ty2 | AE014613 | GenBank | yes | yes |
| Typhi | CT18 | AL513382 | GenBank | yes | yes |
| Typhimurium | LT2 | AE006468 | GenBank | yes | yes |
| Typhimurium | 14028S | CP001363 | GenBank | yes | yes |
| Typhimurium | D23580 | FN424405 | GenBank | yes | yes |
| Typhimurium | SL1344 | FQ312003 | GenBank | yes | yes |
| Uganda | R8-3404 | AFCV00000000 | Internal sequencing | yes | yes |
| Urbana | R8-2977 | AFCW00000000 | Internal sequencing | yes | yes |
| Virchow | SL491 | ABFH00000000 | GenBank | No | yes |
| Wandsworth | A4-580 | AFCX00000000 | Internal sequencing | yes | yes |
| Weltevreden | HI_N05-537 | ABFF00000000 | GenBank | No | yes |

TABLE 2
52 SNPs, shown in lowercase, with flanking sequences of 100 nucleotides on
both sides. Coordinates refer to S. Enteritidis genome

| | Flank- | Flank- | | |
|---|---|---|---|---|
| | ing | ing | | |
| SNP | left | right | | |
| coor- | coor- | coor- | | SEQ ID |
| dinate | dinate | dinate | Sequence | NO |
| 104534 | 104434 | 104634 | TGATCGGCGACGGCTTAAGCAGAATATGACGAGCGTGAACTTCGGTCACGGAGATACTCTGGC | SEQ ID |
| | | | TCTGACCGCGCAGGTCGTTTACTTTCAGAATGTGGAAaCCGACGCCGGAGCGAATCGGGCCGA | NO: 1 |
| | | | CAATGTCGCCTTTCTTCGCGGTGCTCAGCGCCTGGGCGAAAATCCCCGGCAGCTCCTGGATAC | |
| | | | GGCCCCAGCCCA | |
| 104819 | 104719 | 104919 | CAATGCTTTCCGCCTGGCGCTGTGCGTCGTTAACCTGCTCGGAGGTTGGGTTTTCCGGCAGAG | SEQ ID |
| | | | CAATCAGGATATGGCTCAGGTTCAGCTCGGTGCTGGCgTCGTTTTGGGTGCCAATCTGTTTTG | NO: 2 |
| | | | CCAGCGCGTCAACTTCTTGCGGCAAAACGGTGATACGGCGACGAACCTCGTTGTTGCGCACTT | |
| | | | CAGAGATAATCA | |
| 141238 | 141138 | 141338 | GAAGCTGCCACTCTGCTTGTTCATTTGCATCATTTTAACGGCGGTGACGGTGGTCACGACGGC | SEQ ID |
| | | | GCACCATACTCGTTTACTCACCGCGCAGCGTGAACAAtTGGTTCTGGAGCGCGATGCATTGGA | NO: 3 |
| | | | CATTGAATGGCGCAACCTGATCCTTGAAGAAAATGCGCTAGGCGATCACAGCCGGGTGGAGCG | |
| | | | GATCGCAACGGA | |
| 343457 | 343357 | 343557 | GCTATTTCGCGCCGCGCGGTATCGCGATGTTAACGCTTGATATGCCTTCGGTTGGATTTTCAT | SEQ ID |
| | | | CAAAGTGGAAATTAACCCAGGATTCCAGCTTGCTCCAcCAGCATGTGTTAAAAGCGCTTCCTA | NO: 4 |
| | | | ATGTTCCCTGGGTGGATCATACCCGTGTTGCGGCGTTTGGTTTCCGTTTTGGCGCCAATGTTG | |
| | | | CGGTGCGTCTGG | |
| 579050 | 578950 | 579150 | AGCCGCGTGCGACGCACCATATTCAGGAAATTATCGAGCTCACCCGCACCTTAATCGAGAAAG | SEQ ID |
| | | | GACACGCCTATGTGGCGGATAACGGCGATGTGATGTTCGATGTACCGACCGATCCGACTTATG | NO: 5 |
| | | | GGCAGCTTTCCCGTCAGGATCTTGAGCAGCTTCAGGCGGGCGCGCGCGTAGATGTCGTTGACG | |
| | | | TGAAGCGTAACC | |
| 680692 | 680592 | 680792 | ACGAACGGTACACTTTCGTGCTGATAGGATAGATGTCATGGCCCGGAATCAATACCGAGGCAT | SEQ ID |
| | | | TGACGGTCATCACCATCTGATATTCCGCCGTCTGGCCgTCCTGGAACACTGACGCCGTATCCT | NO: 6 |
| | | | GCGAAATAGTCACCGTTCCAAGCCGCAGAGACGGAACGTCTTTGCGCGTTGTGTCTTTATCAA | |
| | | | GCAAGTTTACGT | |
| 869374 | 869274 | 869474 | ATTCGAAATAAATTTGTGGTTCTGCTCACATTGTTATTAAAATGTTTCTGGTCGCAGTTACAG | SEQ ID |
| | | | CCAGGAGCTTAAGTATGACCGTTAACGTTGTCGTTACcGATATGGACGGCACTTTTCTCGACG | NO: 7 |
| | | | ATGCCAAGCAGTACGATCGTGTACGCTTCATGGCGCAATACCAGGAACTGAAAAAACGTAATA | |
| | | | TCGAATTTGTAG | |
| 869620 | 869520 | 869720 | AGCTGAAAGACGAAATTTCCTTTGTCGCAGAAAATGGCGCGCTGGTGTATGAACATGGCAAGC | SEQ ID |
| | | | AGTTGTTTCACGGGGAGCTAACCCAACATGAATCGCGtATTGTGATTGGCGAACTGCTGAAGG | NO: 8 |
| | | | ATAAGCAACTTAATTTTGTCGCCTGCGGTCTGAAAAGCGCTTATGTCAGCAAAAACGCTCCCG | |
| | | | AGACTTTCGTGG | |
| 872425 | 872325 | 872525 | TTTCTTCACTTACGGAAAAACCTGCGCCGGGCCGGTCTGCCAGGTCGTCGATCTCTTTTTCAA | SEQ ID |
| | | | TCCACGAGACGGTATATTCCGGGAAAATCGGCGCGGCgCGGACTTCACTTGCCTGGTTGCCGA | NO: 9 |
| | | | CGATCAGTTCATCGTGTTTTATCCAGATAGTGCGTTCCGCCAGGTGATGGGCCAGCGCCAGCG | |
| | | | CGCGGCGTACCG | |
| 874018 | 873918 | 874118 | TCGCATCGTACATGACGATTTTTCCGCTAGCGGGCTGACCGTAATGCGCCAGCAGCTTAATGG | SEQ ID |
| | | | CCTCCATTGCCTGTAGCGAACCGATCACGCCAATCAGtGGCGCCATCACCCCTGCCTCTACGC | NO: 10 |
| | | | AGGTCAGAGCGTTTTCGCCAAACAGACGGCTCAGGCAGCGGTAACAGGGTTCGTTTTCCCGAT | |
| | | | ACGTAAAGACGG | |
| 914443 | 914343 | 914543 | GGCAGGATCGCTTTATTCCTAACGCTATACCGGCTGTGGATGTTGCTGCTTTATTACCTTTTT | SEQ ID |
| | | | CGCCGAACGCGGCCACGCTGGGATTCGTCTTTTGTACtTTCGGCACGATTTTTTCCATGGGCA | NO: 11 |
| | | | TCCTGCTGCTCATCCATAGCCCTATTATGGTATTGCCTGGTTTTGTTCCGCTCTTCTTTTCCG | |
| | | | GCGGTCCCATCG | |
| 1085897 | 1085797 | 1085997 | GAACGGCGGCAAGCTGCGTCAACGCGTCCTGTTCGTCATCAGCCAGGCATAATACCCGTTCAC | SEQ ID |
| | | | GCGGCAACAGCGTCCAGGTATTGCGCTCGCCGGTCGGtCCCGGTAGCAGGCGCTGCGTGCCGG | NO: 12 |
| | | | CCTGCGCCAGATCGGCGAATTGTCGGCAGAGCGTCTGTAGCGCCGGGCGATCCGCCGCCCATT | |
| | | | GCGTCAGAGCGG | |
| 1090910 | 1090810 | 1091010 | TGTTTAACCCGTGGATTGCCGGTGTTCTGCTGTCTGCTATCCTGGCGGCGGTGATGTCGACGT | SEQ ID |
| | | | TGAGCTGTCAGTTGCTGGTATGCTCCAGCGCGATTACaGAAGATTTATATAAGGCTTTTCTGC | NO: 13 |
| | | | GTAAAAGCGCCAGCCAGCAAGAGCTGGTATGGGTAGGGCGAGTGATGGTGCTGGTGGTAGCGC | |
| | | | TGATCGCCATTG | |
| 1317814 | 1317714 | 1317914 | CCGCAGGCGCCGCGCTATATCTGGAAGCTTCAGCAGGCGCTGGACGCCATGTAGTTTAGCGTG | SEQ ID |
| | | | AAGATTATTCTCGCACTTTATTCGCTCTTTCTCGGGCcTCGCGCTCAAGCGAAAAAATAATCC | NO: 14 |
| | | | GCTGCAACTCCCGCTCTACCGCCGGGCTGACGTTAAGGAAACGAAAGCTCAGGCGGGGAGTGG | |
| | | | TGATCGTTTCAT | |
| 1341676 | 1341576 | 1341776 | ACCGAACCCGCGAGCGCGCGCAAGCCCTGGCGGATGAGGTAGGCGCTGAGGTTATCTCGCTCA | SEQ ID |
| | | | GCGATATCGACGCCCGCTTGCAGGATGCCGATATTATcATCAGTTCGACCGCCAGCCCGCTGC | NO: 15 |
| | | | CGATTATCGGTAAAGGCATGGTGGAGCGCGCATTAAAAAGCCGTCGCAACCAGCCGATGCTGC | |
| | | | TGGTGGATATCG | |
| 1586827 | 1586727 | 1586927 | GATTAGAGAGCGGCATGACGATCGGGCGAGGGCAATGCTTATGCATCTCGCGGATGATCTCTT | SEQ ID |
| | | | CAGTAAAGAGCCCGGTCTGGCCGGAAACCCCGATCAGgATATCCGGCTTCACGTTACGCACCA | NO: 16 |
| | | | CATCCAGCAGCGACAGCACGTCATTTTCGGTATCCCAGTGCTGGAGATTGTCGCATTTCTGCA | |
| | | | CCAGTTTTGCCT | |
| 1673378 | 1673278 | 1673478 | CCGTAACCTTACGTTCGGCAAGTTTCTCCATTAACCCAGGATTTTGCGCAGGCCAGATAAAAC | SEQ ID |
| | | | TGACCAGCGTAGTCCCGGGGTTCAGTAAAGCGATTTCcTCTTCTTCAGGCGCATTAACCTTGA | NO: 17 |
| | | | GAATAATTTCCGACTGCCAGATAGCATTGCCGTCTACAATATCCGCGCCAGCCTGCGCAAACG | |
| | | | CTTTGTCGTCAA | |
| 2199971 | 2199871 | 2200071 | TCATTAGCACTGCGAACCAGGAAAAATAAATAATCAGCAGATACAATCCATTGTCTATGGTTT | SEQ ID |
| | | | TCCCGACATCCGCACCGTTAAAGATTCCGAATGATGCgACATATTCATAAAGTGAGCCAAATC | NO: 18 |
| | | | TGACTACACCATCAATATGGGTCAAGGAATATCCGACCATGACTAAGGGGCCCACAATACGAT | |
| | | | AATAAGAAGATG | |
| 2207082 | 2206982 | 2207182 | ACATCGCCAGGCCCGACGCGATACTGATAGCTGGCGATCTCCTGGTCCAGCGACATATTCGGT | SEQ ID |
| | | | TGCGCGACATTGGGCCGCGGGCGTAATTGCTCAACCAgCCGTGGCGTCAGCGGATACACATTG | NO: 19 |
| | | | ACCATCCGGTCGAGATCAAAGTCAGCGTCTTGCTGTTTGATCACATCTTTCCCCATCGTAGAC | |
| | | | ATATTGCTGCCC | |
| 2207589 | 2207489 | 2207689 | CGCATTCCCCTCAGATTTTATCCTCGTTCAGATAAAAATCTTCAGCGAAATAAAGGTTACGTA | SEQ ID |
| | | | TTAATAGTTTTCTCGATGTCGTGACAGGATATTTAAGaGCGCTATCTTAATCCTGCAAGAATT | NO: 20 |
| | | | GTTGCAGCTAACCTAATGACAGGCAAGGTAACAAACCCGCCATTCTATATTTTTAAGATTAAT | |
| | | | ATTAAATGAATT | |
| 2243744 | 2243644 | 2243844 | GCGCCTCTTTCAGGGAATATTCAAAGATATTATTGATTTGCGTACCGTAGCCGCGGGCGCGCG | SEQ ID |
| | | | TGACGACCCCCTCTTCTTCCAGCGCCTGCATCGCTTTtCGTACCGTGATGCGCGAGACGCCGG | NO: 21 |
| | | | TAAGCTGGCTGAGATCGCGTTCGCCAGGCAAAATATTACCGTGCTCAAGAATACCGCTGCGCA | |
| | | | CGGCATCCTTTA | |
| 2300257 | 2300157 | 2300357 | TATCGCCCACGTTCAGAACGCCCGCACGCAGTTTAACGTTTTTCGTCGCCTGCCATGCCGCGC | SEQ ID |
| | | | CGGTATCCCAGACCACGTACCCGCCCGGCGTTTTCGCtGTTGCGCTGTCGGCCCGCTTACGCC | NO: 22 |
| | | | CGGTATACTTCCCTGATACGTAGAATGACCAGTCTTCGAGCTGCGCCGGTTTCCAGTCCAGCG | |
| | | | TACCGTTCGCGG | |
| 2302792 | 2302692 | 2302892 | CGCGACGGAAGCGATAATGGCTAATGGCAATCCCCAGCCAGGCGATGAAGCCCGTCATACCGG | SEQ ID |
| | | | AGGTGTTCAACAGCCACAGATAGACCGTCTGGTTGCCaAACATTGAGGTCAAAAAGCAAAGAC | NO: 23 |
| | | | CGGCAATCACGGTGGTGGCATAAAGCGCATTGCGCGGTACGCCGCCGCGGGACAATTTCGCAA | |
| | | | AAATACGCGGGG | |
| 2309364 | 2309264 | 2309464 | TGCATGGACACTGGCTGCGCAGACGCGTCATCCAGTCGGTGAACGCTTCCGGACTCACGCCAG | SEQ ID |
| | | | CCGGTAAGCTACCGCTGACGCAGACCATATCGAACTGgCCCAGCCAGCTCAGGGAGTCGTTAA | NO: 24 |
| | | | CAAAGCGTTCCCAGTCTGCGGGGGTCACGTCAAAGCCGGAAAAGTTGAAGTCGGTCACTTCGC | |
| | | | CATCTTTTTCCG | |
| 2326486 | 2326386 | 2326586 | CAAAACGGCTGTTGATAATCGTCATCACAAACAGAAATACGATGTTCAACGCGAAGTAGTAGC | SEQ ID |
| | | | CGAAATCCTGCGGCGGAACATGATTAATTTCGATATAaACAAACGGCCCCGCGCTCAAAAAAG | NO: 25 |
| | | | AGAACATACCGGCAAAACTAAACCCGCTCGCCAGCATATAGCTCAGTACGCGTTTGTGGCGAA | |
| | | | ACAACGCGGCAA | |
| 2433579 | 2433479 | 2433679 | AATGGCAACGATAAGAAAGATAGCGAATGCCCAGTGATGAGCGATGACTTCAGTGGATGTTGA | SEQ ID |
| | | | CATACTCATTGCTTACTCATCAAAAGTAGCGCCAGTTcCTCTGCTCTTTACGGCAGATGGACG | NO: 26 |
| | | | CCACATCGATTCATGGGGAGGAAT CCCTACAATTACTGTAGAAAATGATAAAAAC | |
| | | | AGCTAATTGATG | |
| 2433757 | 2433657 | 2433857 | ATGATAAAAACAGCTAATTGATGTGGTTTTTTACTCCTTTCTATAACCTTTTGTCAACTTTAA | SEQ ID |
| | | | CAAAAGTTCCTTCACATTAATTTACATCAATTCATCAtCATTAACATTAAGTGCCATCCTGGA | NO: 27 |
| | | | GAAAATGACTCTTCGACCAGGGGGGATTTTATGTTGTTGAATCGTGTCCGTTGTGAAGCAAAA | |
| | | | GAAATAACACAC | |
| 2441456 | 2441356 | 2441556 | TAAGGATTGAAGGCGGCAACGGCGTCACGGAACGTTTCCCTAACCACCATAATCCCTGCATAG | SEQ ID |
| | | | GCAAGCTCAGGGCGAACAGGGCGGTCGCCACGGCCGGaCCTAACTGACCGCCCAGCGCTATCT | NO: 28 |
| | | | GCCAGCATAATGTAAAGACGGCGACAGGCGGCATAAATCGTATGGCATAGCGCGTCATACGAA | |
| | | | TGACGCGGTTTT | |
| 3107015 | 3106915 | 3107115 | CGGACCAGCTCAGCACCTCATCGCGCTGGTCGAGTGGGTGACACCAAACCAGCGAACATCGGC | SEQ ID |
| | | | CATGCGACATCGGCAACATCGCCAGCGGGCCATTGGGaGTAAAACGTTCAAAGGCGCGTCCCT | NO: 29 |
| | | | GATGCGGGATAGCGGTCGCGACATTCGCTATCACCGCTAACTGTTCGTAAGGCTCCTGATGCC | |
| | | | AGTCAACGCCGC | |
| 3144381 | 3144281 | 3144481 | GCAGTAATCAGGTATTCGATGCTGAAATCATTAGCGCCAGTAAGAAAAGCGTTGAAGTGCAAG | SEQ ID |
| | | | TGATGAAAGGCGAAATCGACGATCGTGAATCGCCGCTaCATATCCATCTGGGCCAGGTGATGT | NO: 30 |
| | | | CGCGCGGTGAAAAAATGGAATTTACTATCCAGAAATCGATCGAACTAGGTGTAAGCCTCATTA | |
| | | | CGCCACTGTTCT | |
| 3151178 | 3151078 | 3151278 | CTAAACTACCGCCGCTCAGTCTTTATATTCATATTCCCTGGTGTGTACAAAAATGTCCATATT | SEQ ID |
| | | | GCGACTTCAATTCCCATGCGTTGAAGGGCGAGGTGCCaCATGACGACTACGTCCAGCATCTGT | NO: 31 |
| | | | TAAACGATCTGGATGCCGACGTCGCTTGGGCGCAAGGGCGTGAAGTAAAGACCATTTTTATTG | |
| | | | GCGGCGGTACGC | |
| 3151680 | 3151580 | 3151780 | GGACCAAACGCTGGAAGAGGCGCTGAATGATTTGCGACAGGCAATTGCGCTTAATCCGCCGCA | SEQ ID |
| | | | TCTCTCATGGTATCAATTGACGATTGAACCCAACACToTGTTCGGTTCGCGTCCGCCGGTTTT | NO: 32 |
| | | | ACCGGACGATGACGCTCTGTGGGATATCTTTGAGCAGGGCCACCAGTTATTAACCGCCGCGGG | |
| | | | CTATCAGCAATA | |
| 3355747 | 3355647 | 3355847 | TTCTTGCCGGTTGTACGCCTGAGCAAGAGAAAGGGTTGCAGGACTATGGCCGCTACCTTGGTA | SEQ ID |
| | | | CGGCTTTCCAGCTCATTGACGATCTGCTGGATTACAGtGCCGACGGCGAGCATCTCGGTAAAA | NO: 33 |
| | | | ATGTGGGTGATGACCTCAATGAGGGCAAACCTACCTTACCGTTGCTTCACGCCATGCGCCACG | |
| | | | GTACGCCAGAAC | |
| 3425000 | 3424900 | 3425100 | TGTACTGCCCCAGCATCAGCAGGTCGCTGTTTTTTAGCAGTAATTCCTGGCGCAGCGCCCGAT | SEQ ID |
| | | | TTTCCAGCTCAAGCTGGTCGCGAGAAGCCAGCGTTTGoGACACGCTGTCGAGCAGTTCACGAG | NO: 34 |
| | | | GGCCGTTTGAAATAAAATAGAAAGGACTGACGGCAGTGTCCATGTACGTTCGGATTTGACTGA | |
| | | | ACGTCCCCAGGC | |
| 3498114 | 3498014 | 3498214 | TGGCTGTCGGTTTATTGATAGGCCAGCTACACCTGGGCTTGCTTTTCTCTCTCGTTCCCGCCT | SEQ ID |
| | | | AAAA AAA | NO: 35 |
| | | | CGTCGCTGTTCGCCGGATGCAGTCTGGTAACGCAACTTCTGCTGGCGGAATCCATCCCCCTGC | |
| | | | CGTTGATCCTGA | |
| 3501524 | 3501424 | 3501624 | TACTTTCCGGGTGGAACTGCACGCCTTCCAGATCCCACTCGCGGTGGCGAATGCCCATAATCT | SEQ ID |
| | | | CCTGCGTTTCGCTCCAGGCGGTGATCTCAAAACACTCaGGCAACGTGGCAGGGTCGACAATCA | NO: 36 |
| | | | GCGAGTGGTAACGTGTCACGGTTAACGGGCTGGGCAATCCCCGAAACACGCCCTGTCCATTAT | |
| | | | GCGTAACAGGTG | |
| 3776636 | 3776536 | 3776736 | CAAGCATCATGCCAAAACGACGGTTGCGCGACTGGTTGTCGGTACAGGTCAGCACCAGATCGC | SEQ ID |
| | | | CTAAACCCGCCATCCCCATAAAGGTGGCGGGATCGGCaCCAAGCGCTGCGCCAAGCCGCGACA | NO: 37 |
| | | | TTTCGGTCAGTCCACGCGTGATTAGCGCCGTGCGGGCGTTCGCGCCGAAGCCGATGCCGTCAG | |
| | | | ACATCCCCGCGC | |
| 3778064 | 3777964 | 3778164 | TGATCATCTCTTCACGCTTCACGGCGTCGCCGTCAATCGCAATTTCCTGGAAACTCACGCCCT | SEQ ID |
| | | | TGCTGTTTAACAGCGCCTTTGCACGATGGCAAAACGGgCAGGTTGCTTTGGTGTAGATTTCAA | NO: 38 |
| | | | TATTGGCCATGACTTCGCTCCTGTTTTTTTACCCGATGGATTTCATGTCGACGCAAAGGCAAG | |
| | | | TCATTCCCCTTG | |
| 3866852 | 3866752 | 3866952 | ACCGCGGCTGTTTTGTATTTTCAATCCTTCCACGCCGCTGGCGTAACGAAACGCCGTAACCGT | SEQ ID |
| | | | AAAATCGTCATTTTCCAGCAAGATACGCGGCTGCTCGcTAAAAAGCTCCCGCCATAATGTAAT | NO: 39 |
| | | | ACGTATTGTCATGGTTGATCTCCTCAGGACGCGGCGACTTCAGCCAGGTTGCCGCGCACTTTG | |
| | | | CTTTCGCGCCAG | |
| 3867081 | 3866981 | 3867181 | GCATGGAAACCAGGAATGAGAGCTGTAGCGAGTGGAACATGTCCGCCACGTAGCCCTGAATCG | SEQ ID |
| | | | CGGGAACCACCGCCGCGCCGACAATCGCCATCACGATgACCGCCCCCGCCATTTCGGTATGCT | NO: 40 |
| | | | CGTTATCGACGGTATCGAGCGTTCCGGCATAAATGGTCGCCCAGCAGGGGCCAAACAGAACAC | |
| | | | TGACCAGCACCG | |
| 4002077 | 4001977 | 4002177 | GTGATGCTGGTCGATCACGACGAATTTAAAGCGATTCCCGGCGATGCGGTTCATCAACGTTAT | SEQ ID |
| | | | GTGGTTGATACCAAAGGAGTCTGGCGCTGATGAAACGcATTCTGGTGACCGGAGGCGCAGGCT | NO: 41 |
| | | | TTATCGGATCTGCGGTGGTGCGGCATATCATCCATGAAACGGCAGACGCGGTGGTGGTGGTGG | |
| | | | ATAAACTCACCT | |
| 4129096 | 4128996 | 4129196 | ATGAGCGCCCATACAGAATTACCCGCGGCGATCTGTTTTACATTCGTGCTGAAGACAAACACT | SEQ ID |
| | | | CCTATACCTCCGTTAACGATCTGGTGCTGCAAAATATcATTTACTGCCCGGAGCGTTTGAAAC | NO: 42 |
| | | | TCAATGTAAACTGGCAGGCGATGATTCCCGGCTTTCAGGGAGCGCAGTGGCATCCTCACTGGC | |
| | | | GGCTGGGCAGTA | |
| 4129999 | 4129899 | 4130099 | ACTGCAAATACCACATCAGACCGCCAAGCGCGGACAGCAGAATATTGCTGATAATCAACGGTC | SEQ ID |
| | | | TTGCCAGCGAGAAGTCGGCTTTTATCGACAGATTTTGtACTTTTGCCAGGCGGATAAAACAGA | NO: 43 |
| | | | AACCGAGGTTAACCAGCGCGCCGCCGCCCATAATCACCACGTAACTCGGCAGCGCGACATAGA | |
| | | | GCGGGTCAACGC | |
| 4191971 | 4191871 | 4192071 | GCTGAGATATGAGCACGACCGACGATACCCATAACACGTTATCCACTGGAAAATGTCCTTTCC | SEQ ID |
| | | | ATCAGGGGGGGCATGACCGAAGCGCAGGCGCAGGGACtGCCAGCCGCGACTGGTGGCCGAACC | NO: 44 |
| | | | AGCTTCGCGTGGATCTTTTGAATCAACATTCCAACCGTTCTAACCCGCTGGGTGAAGACTTTG | |
| | | | ACTACCGCAAAG | |
| 4206466 | 4206366 | 4206566 | AGGTTTTATACCCGGCCTGTTTCATCATATTCATCAGCGATGGCTTCGTCAGATACCAGTCCG | SEQ ID |
| | | | GGTTCTTTTCGTCCGCGAACGTTAGCGCCTGTTGCAGgATCTCAATGGTGTACGGTCGCGAAG | NO: 45 |
| | | | TTACCACATTATTAAACACGGTCAGCCCGGGGTCGGTTTTATGCAGCGCGTCCAGTTCCGGCG | |
| | | | TGGTTTCGCGCG | |
| 4206625 | 4206525 | 4206725 | CGGTTTTATGCAGCGCGTCCAGTTCCGGCGTGGTTTCGCGCGGATAACCGTACAGACTCATAC | SEQ ID |
| | | | GGCCACGCTGGGTCGATTCGCCAATCACCAGTACCAGtGTGCGCGGCGCATCGCCGGAGTGGT | NO: 46 |
| | | | CCTGGAAGTTAGCCAGCGGCGGCAGGGCATCGTTTTCATTCAGCAGTTTATTCAGCGAGGCGA | |
| | | | GCTGTAAACGGT | |
| 4207216 | 4207116 | 4207316 | CAATAACGGCCGCAATAACCCGGATGCGGCCTGGAAACAGAAAGACCGGGATCAGCCACAGTG | SEQ ID |
| | | | AGCTGTACAGTAGCGAATCCCGCAGACCGTTTGTCCCgCTGTATCCGGTGAGATAAATAATGG | NO: 47 |
| | | | CCTGCAGGAGGGTGGAAAAGAACCAAAAGTAGAGCAGTGCCCAGCCCAGGGCTTTCCAGCTAA | |
| | | | ACGCGGGTTTAG | |
| 4207666 | 4207566 | 4207766 | TACCGGCGGCGACGCCCGCAATCGTGACCATCAACGCCTGTTCGACGCGAGGATCGGGCGATT | SEQ ID |
| | | | TGCCCTGCTCTTCAGTCAGACGGGAACGATACAGCAAcTCGGCCTGCAACACGTTTAATGGAT | NO: 48 |
| | | | CGGTATAAACGTTTCTTAACTGAATGGACTCCGCAATCCACGGTAAGTCGGCCATCAGGTGCG | |
| | | | AATCGTTGGCAA | |
| 4240670 | 4240570 | 4240770 | AGCGTGTTGTCCAGGAAGACCGTTTCACCACCATCCACATTCAGGAACTGGCGTGCGTGTCCC | SEQ ID |
| | | | GTGACACCAAGCTGGGGCCGGAAGAGATCACCGCTGAcATCCCGAACGTGGGTGAAGCTGCGC | NO: 49 |
| | | | TCTCCAAACTGGATGAATCCGGTATCGTTTACATTGGCGCGGAAGTGACCGGCGGCGACATTC | |
| | | | TGGTCGGTAAGG | |
| 4244367 | 4244267 | 4244467 | ATATCTGGGCTGCGGCGAACGATCGTGTATCTAAAGCGATGATGGATAACCTGCAAACTGAAA | SEQ ID |
| | | | CCGTGATTAACCGTGACGGCCAGGAAGAGCAGCAGGTcTCCTTCAACAGCATCTACATGATGG | NO: 50 |
| | | | CCGACTCCGGTGCGCGTGGTTCTGCGGCACAGATTCGTCAGCTTGCTGGTATGCGTGGTCTGA | |
| | | | TGGCGAAGCCGG | |
| 4245891 | 4245791 | 4245991 | AATGGCGTCAGCTCAACGTGTTCGAAGGGGAACGTGTAGAACGTGGTGATGTGATTTCCGACG | SEQ ID |
| | | | GTCCGGAAGCGCCGCACGATATTCTGCGTCTGCGTGGtGTTCATGCTGTGACGCGTTACATCG | NO: 51 |
| | | | TTAACGAAGTCCAGGATGTATACCGTCTGCAGGGCGTTAAGATTAACGATAAACACATCGAAG | |
| | | | TTATCGTTCGTC | |
| 4372200 | 4372100 | 4372300 | GGAGGTATAAAGGATAAACAGCGGATCTTCGGCATTCATCGCTTCAGGCTGGTGCTCAGGGCT | SEQ ID |
| | | | GGCTTTTTCAATCAAATCGCGCCACCACAGGTCGCGGcCTTCTTGCCAGTCAATGTCGCTGTC | NO: 52 |
| | | | GGTGCTCTTCAGGACGATCACATGCTCAACGCTAGTGACATTCGGGTTTTTCAGCGCGTCATC | |
| | | | GACATTCTTTTT | |

TABLE 3
Primer and probe sequences. Coordinates refer to S. Enteritidis genome.

| S. | | | | |
|---|---|---|---|---|
| Enteritidis | | | | |
| coordinate | Forward primer | Reverse primer | Probe 1 (FAM-MGB) | Probe 2 (VTC-MGB) |
| 104534 | ACGGAGATACTCTGGCTCT | CATTGTCGGCCCGATTCG | TGTGGAAgCCGACGCC | ATGTGGAAaCCGACGCC |
| | GA [SEQ ID NO: 54] | [SEQ ID NO: 106] | [SEQ ID NO: 158] | [SEQ ID NO: 210] |
| 104819 | CCGGCAGAGCAATCAGGAT | GCGCTGGCAAAACAGATTGG | CCCAAAACGAtGCCAGC | CCAAAACGAcGCCAGC |
| | ATG [SEQ ID NO: 55] | [SEQ ID NO: 107] | [SEQ ID NO: 159] | [SEQ ID NO: 211] |
| 141238 | GCGCACCATACTCGTTTAC | GTTGCGCCATTCAATGTCCAA | CCAGAACCAgTTGTTCA | CCAGAACCAaTTGTTCA |
| | TCA [SEQ ID NO: 56] | [SEQ ID NO: 108] | [SEQ ID NO: 160] | [SEQ ID NO: 213] |
| 343457 | CAAAGTGGAAATTAACCCAGG | GCCGCAACACGGGTATGA | CTTTTAACACATGCTGgTG | CTTTTAACACATGCTGa |
| | ATTCC [SEQ ID NO: 57] | [SEQ ID NO: 109] | GA [SEQ ID NO: 161] | TGGA |
| | | | | [SEQ ID NO: 214] |
| 579050 | CGACGCACCATATTCAGGA | CAAGATCCTGACGGGAAAGCT | CGGTACATCgAACATC | CGGTACATCaAACATC |
| | AATT [SEQ ID NO: 58] | [SEQ ID NO: 110] | [SEQ ID NO: 162] | [SEQ ID NO: 215] |
| 680692 | CCGAGGCATTGACGGTC | CAGGATACGGCGTCAGTGTT | TCTGGCCaTCCTGG | TCTGGCCgTCCTGG |
| | AT [SEQ ID NO: 59] | [SEQ ID NO: 111] | [SEQ ID NO: 163] | [SEQ ID NO: 216] |
| 869374 | CCAGGAGCTTAAGTATGACCG | TGCGCCATGAAGCGTAC | TTGTCGTTACaGATATGG | TTGTCGTTACcGATATGG |
| | TTA [SEQ ID NO: 60 ] | [SEQ ID NO: 112] | [SEQ ID NO: 164] | [SEQ ID NO: 217] |
| 869620 | CGCGCTGGTGTATGAACA | TCCTTCAGCAGTTCGCCAAT | TGAATCGCGgATTGTG | ATGAATCGCGtATTGTG |
| | TG [SEQ ID NO: 61] | [SEQ ID NO: 113] | [SEQ ID NO: 165] | [SEQ ID NO: 218] |
| 872425 | CTCTTTTTCAATCCACGAGAC | CGGCAACCAGGCAAGTG | AAGTCCGgGCCGCG | AAGTCCGcGCCGCG |
| | GGTAT [SEQ ID NO: 62] | [SEQ ID NO: 114] | [SEQ ID NO: 166] | [SEQ ID NO: 219] |
| 874018 | TCCATTGCCTGTAGCGAACC | GCCGTCTGTTTGGCGAAAA | TGGCGCCgCTGATT | TGGCGCCaCTGATT |
| | [SEQ ID NO: 63] | [SEQ ID NO: 115] | [SEQ ID NO: 167] | [SEQ ID NO: 220] |
| 914443 | TGTGGATGTTGCTGCTTTATT | GAAAAGAAGAGCGGAACAAAA | TCGTGCCGAAgGTACAA | CGTGCCGAAaGTACAA |
| | ACCT [SEQ ID NO: 64] | [SEQ ID NO: 116] | [SEQ ID NO: 168] | [SEQ ID NO: 221] |
| 1085897 | CCGTTCACGCGGCAA | CCGACAATTCGCCGATCTG | CGGTCGGcCCCGGT | CGGTCGGtCCCGGT |
| | [SEQ ID NO: 65] | [SEQ ID NO: 117] | [SEQ ID NO: 169] | [SEQ ID NO: 222] |
| 1090910 | GCTGTCAGTTGCTGGTATGCT | CTGGCGCTTTTACGCAGAAA | CGCGATTACaGAAGAT | CGCGATTACgGAAGAT |
| | [SEQ ID NO: 66] | [SEQ ID NO: 118] | [SEQ ID NO: 170] | [SEQ ID NO: 223] |
| 1317814 | CGCTGGACGCCATGTAGTTTA | GGGAGTTGCAGCGGATTATTT | TTTCTCGGGCcTCGCG | TTTCTCGGGCgTCGCG |
| | [SEQ ID NO: 67] | [SEQ ID NO: 119] | [SEQ ID NO: 171] | [SEQ ID NO: 224] |
| 1341676 | AGCGCGCGCAAGC | TGCGCGCTCCACCAT | AGGATGCCGATATTATtAT | ATGCCGATATTATcAT |
| | [SEQ ID NO: 68] | [SEQ ID NO: 120] | CA [SEQ ID NO: 172] | CAGT |
| | | | | [SEQ ID NO: 225] |
| 1586827 | TCGCGGATGATCTCTTCAGTA | GATGTGGTGCGTAACGTGAAG | CCGATCAGaATATCCG | CGATCAGgATATCCG |
| | AAG [SEQ ID NO: 69] | [SEQ ID NO: 121] | [SEQ ID NO: 173] | [SEQ ID NO: 226] |
| 1673378 | ACGTTCGGCAAGTTTCTCCAT | ATTCTCAAGGTTAATGCGCCT | AAGCGATTTCaTCTTCT | AAAGCGATTTCcTCTTCT |
| | TAA [SEQ ID NO: 70] | GA [SEQ ID NO: 122] | [SEQ ID NO: 174] | [SEQ ID NO: 227] |
| 2199971 | GACATCCGCACCGTTAAAG | GGTCGGATATTCCTTGACCCA | CGAATGATGCgACATAT | CGAATGATGCaACATAT |
| | ATTC [SEQ ID NO: 71] | TATT [SEQ ID NO: 123] | [SEQ ID NO: 175] | [SEQ ID NO: 228] |
| 2207082 | CCTGGTCCAGCGACATATTCG | AGCAAGACGCTGACTTTGAT | TTGCTCAACCAaCCGTG | TTGCTCAACAgCCGTG |
| | [SEQ ID NO: 72] | CT [SEQ ID NO: 124] | [SEQ ID NO: 176] | [SEQ ID NO: 229] |
| 2207589 | GTTACGTATTAATAGTTTTCT | TGTCATTAGGTTAGCTGCAAC | ATAGCGCgCTTAAAT | AGATAGCGCtCTTAAAT |
| | CGATGT [SEQ ID NO: 73] | AATTC [SEQ ID NO: 125] | [SEQ ID NO: 177] | [SEQ ID NO: 230] |
| 2243744 | TTCTTCCAGCGCCTGCA | CGCGATCTCAGCCAGCTTA | CGGTACGgAAAGCGA | CGGTACGaAAAGCGA |
| | [SEQ ID NO: 74] | [SEQ ID NO: 126] | [SEQ ID NO: 178] | [SEQ ID NO: 231] |
| 2300257 | TGCCGCGCCGGTATC | CGTAAGCGGGCCGA | CAGCGCAACgGCGAA | CAGCGCAACaGCGAA |
| | [SEQ ID NO: 75] | [SEQ ID NO: 127] | [SEQ ID NO: 179] | [SEQ ID NO: 232] |
| 2302792 | CCGTCATACCGGAGGTGTT | GTGATTGCCGGTCTTTGCTT | CCTCAATGTTtGGCAACC | CTCAATGTTcGGCAACC |
| | [SEQ ID NO: 76] | TT [SEQ ID NO: 128] | [SEQ ID NO: 180] | [SEQ ID NO: 233] |
| 2309364 | CCGCTGACGCAGACCAT | ACTGGGAACGCTTTGTTAAC | ATCGAACTGaCCCAGCC | CGAACTGgCCCAGCC |
| | [SEQ ID NO: 77] | GA [SEQ ID NO: 129] | [SEQ ID NO: 181] | [SEQ ID NO: 234] |
| 2326486 | ACGATGTTCAACGCGAAGTA | AAACGCGTACTGAGCTATATG | ATTAATTTCGATATAaA | AATTTCGATATAgACA |
| | GTAG [SEQ ID NO: 78] | CT [SEQ ID NO: 130] | CAAACG | AACG |
| | | | [SEQ ID NO: 182] | [SEQ ID NO: 235] |
| 2433579 | AGTGGATGTTGACATACTCA | ATCAATTAGCTGTTTTTAT | AAGAGCAGAGgAACTG | AGAGCAGAGaAACTG |
| | TTGCT [SEQ ID NO: 79] | CATTTTCTACAGT | [SEQ ID NO: 183] | [SEQ ID NO: 236] |
| | | [SEQ ID NO: 131] | | |
| 2433757 | ACTCCTTTCTATAACCTTTT | CAACGGACACGATTCAACAAC | CACTTAATGTTAATGgT | CACTTAATGTTAATGa |
| | GTCAACTTTAACA | ATAA [SEQ ID NO: 132] | GATGAA | TGATGAA |
| | [SEQ ID NO: 80] | | [SEQ ID NO: 184] | [SEQ ID NO: 237] |
| 2441456 | CCTAACCACCATAATCCCTGC | CGATTTATGCCGCCTGTCG | TCAGTTAGGtCCGGCCGT | AGTTAGGcCCGGCCGT |
| | [SEQ ID NO: 81] | [SEQ ID NO: 133] | [SEQ ID NO: 185] | [SEQ ID NO: 238] |
| 3107015 | GGTGACACCAAACCAGCGA | GCGGCGTTGACTGGC | CCTTTGAACGTTTTACt | TTTGAACGTTTTACcCC |
| | [SEQ ID NO: 82] | [SEQ ID NO: 134] | CCCAAT | CAAT |
| | | | [SEQ ID NO: 186] | [SEQ ID NO: 239] |
| 3144381 | TGATGAAAGGCGAAATCGAC | GACATCACCTGGCCCAGAT | AATCGCCGCTaCATAT | CGCCGCTgCATAT |
| | GAT [SEQ ID NO: 83] | [SEQ ID NO: 135] | [SEQ ID NO: 187] | [SEQ ID NO: 240] |
| 3151178 | GACTTCAATTCCCATGCGTT | GCGTACCGCCGCCAATA | AGGTGCCaCATGACG | AGGTGCCgCATGACG |
| | GA[SEQ ID NO: 84] | [SEQ ID NO: 136] | [SEQ ID NO: 188] | [SEQ ID NO: 241] |
| 3151680 | CGCATCTCTCATGGTATCAAT | GGTAAAACCGGCGGACG | AACCGAACAgAGTGTTG | ACCGAACAaAGTGTTG |
| | TGAC [SEQ ID NO: 85] | [SEQ ID NO: 137] | [SEQ ID NO: 189] | [SEQ ID NO: 242] |
| 3355747 | CGCTACCTTGGTACGGCTTT | GCGTGAAGCAACGGTAAGG | CCGTCGGCgCTGTAA | CCGTCGGCaCTGTAA |
| | [SEQ ID NO: 86] | [SEQ ID NO: 138] | [SEQ ID NO: 190] | [SEQ ID NO: 243] |
| 3425000 | TGTACTGCCCCAGCATCAG | TTCAAACGGCCCTCGTGAA | CAGCGTTTGcGACACG | CAGCGTTTGtGACACG |
| | [SEQ ID NO: 87] | [SEQ ID NO: 139] | [SEQ ID NO: 191] | [SEQ ID NO: 244] |
| 3498114 | GCAACATTGCAGGTCTGGA | GCGCCGATGATCAGG | CACAAACGcTTTTTTAA | CACAAACGtTTTTTTA |
| | TAC [SEQ ID NO: 88] | [SEQ ID NO: 140] | ACG | AACG |
| | | | [SEQ ID NO: 192] | [SEQ ID NO: 245] |
| 3501524 | CTCCAGGCGGTGATCTCAAA | ACGTTACCACTCGCTGATTG | CCACGTTGCCtGAGTGT | ACGTTGCCaGAGTGT |
| | [SEQ ID NO: 89] | TC [SEQ ID NO: 141] | [SEQ ID NO: 193] | [SEQ ID NO: 246] |
| 3776636 | CCGCCATCCCCATAAAGGT | CGCACGGCGCTAATCAC | ATCGGCAaCCAAGCG | CGGCgCCAAGCG |
| | [SEQ ID NO: 90] | [SEQ ID NO: 142] | [SEQ ID NO: 194] | [SEQ ID NO: 247] |
| 3778064 | GCCGTCAATCGCAATTTCCT | GCGAAGTCATGGCCAATATTG | AAGCAACCTGtCCGTTTT | CAACCTGcCCGTTTT |
| | [SEQ ID NO: 91] | AAAT [SEQ ID NO: 143] | [SEQ ID NO: 195] | [SEQ ID NO: 248] |
| 3866852 | ACGAAACGCCGTAACCGTAAA | CTGGCGCGAAAGCAAAGT | TGCTCGcTAAAAAG | CTGCTCGtTAAAAAG |
| | [SEQ ID NO: 92] | [SEQ ID NO: 144] | [SEQ ID NO: 196] | [SEQ ID NO: 249] |
| 3867081 | CCGCGCCGACAATCG | TGCCGGAACGCTCGATAC | CCATCACGATcACCGC | CCATCACGATgACCGC |
| | [SEQ ID NO: 93] | [SEQ ID NO: 145] | [SEQ ID NO: 197] | [SEQ ID NO: 250] |
| 4002077 | CCCGGCGATGCGGTT | TGAGTTTATCCACCACCAC | CACCAGAATgCGTTTCA | CACCAGAATcCGTTTCA |
| | [SEQ ID NO: 94] | CAC [SEQ ID NO: 146] | [SEQ ID NO: 198] | [SEQ ID NO: 251] |
| 4129096 | GTTAACGATCTGGTGCTGC | GGAATCATCGCCTGCCAGT | CGGGCAGTAAATgATAT | CGGGCAGTAAATaATAT |
| | AAA [SEQ ID NO: 95] | TTA [SEQ ID NO: 147] | [SEQ ID NO: 199] | [SEQ ID NO: 252] |
| 4129999 | GCCAAGCGCGGACA | GCCGAGTTACGTGGTGATTAT | AAAAGTgCAAAATCT | CAAAGTaCAAAATCT |
| | [SEQ ID NO: 96] | GG [SEQ ID NO: 148] | [SEQ ID NO: 200] | [SEQ ID NO: 253] |
| 4191971 | CCGACGATACCCATAACACG | GGTTCGGCCACCAGTCG | CAGGGACaGCCAGCC | CAGGGACtGCCAGCC |
| | TTA [SEQ ID NO: 97] | [SEQ ID NO: 149] | [SEQ ID NO: 201] | [SEQ ID NO: 254] |
| 4206466 | CCAGTCCGGGTTCTTTTCGT | GAAACCACGCCGGAACTG | CTGTTGCAGaATCTCAA | TGTTGCAGgATCTCAA |
| | [SEQ ID NO: 98] | [SEQ ID NO: 150] | [SEQ ID NO: 202] | [SEQ ID NO: 255] |
| 4206625 | CGCGCGGATAACCGTACA | CGCCGCTGGCTAACTTC | CCAGTACCAGcGTGCG | CCAGTACCAGtGTGCG |
| | [SEQ ID NO: 99] | [SEQ ID NO: 151] | [SEQ ID NO: 203] | [SEQ ID NO: 256] |
| 4207216 | GTGAGCTGTACAGTAGCGAA | GCACTGCTCTACTTTTGGTTC | CCGTTTGTCCCaCTGTAT | CGTTTGTCCCgCTGTAT |
| | TCC [SEQ ID NO: 100] | TTTT [SEQ ID NO: 152] | [SEQ ID NO: 204] | [SEQ ID NO: 257] |
| 4207666 | TCTTCAGTCAGACGGGAACG | TCAGTTAAGAAACGTTTATAC | CAGGCCGAgTTGCT | AGGCCGAaTTGCT |
| | ATA [SEQ ID NO: 101] | CGATCCATT | [SEQ ID NO: 205] | [SEQ ID NO: 258] |
| | | [SEQ ID NO: 153] | | |
| 4240670 | GTCCCGTGACACCAAGCT | GTAAACGATACCGGATTCATCC | ACGTTCGGGATgTCAG | CGTTCGGGATaTCAG |
| | [SEQ ID NO: 102] | AGTT [SEQ ID NO: 154] | [SEQ ID NO: 206] | [SEQ ID NO: 259] |
| 4244367 | GCGATGATGGATAACCTGCA | AATCTGTGCCGCAGAACCA | CTGTTGAAGGAgACCTG | TGTTGAAGGAaACCTG |
| | AAC [SEQ ID NO: 103] | [SEQ ID NO: 155] | [SEQ ID NO: 207] | [SEQ ID NO: 260] |
| 4245891 | TGTAGAACGTGGTGATGTGAT | CCCTGCAGACGGTATACATCCT | CAGCATGAACgCCACGC | CAGCATGAACaCCACGC |
| | TTCC [SEQ ID NO: 104] | [SEQ ID NO: 156] | [SEQ ID NO: 208] | [SEQ ID NO: 261] |
| 4372200 | GGGCTGGCTTTTTCAATCAAA | TGAGCATGTGATCGTCCTGAAG | TGGCAAGAAGgCCGCGAC | TGGCAAGAAGaCCGCGAC |
| | TCG [SEQ ID NO: 105] | [SEQ ID NO: 157] | [SEQ ID NO: 209] | [SEQ ID NO: 262] |

Each embodiment disclosed herein may be used or otherwise combined with any of the other embodiments disclosed. Any element of any embodiment may be used in any embodiment. Although the invention has been described with reference to specific embodiments, it will be understood by those skilled in the art that various changes may be made and equivalents may be substituted for elements thereof without departing from the true spirit and scope of the invention. In addition, modification may be made without departing from the essential teachings of the invention.

1-18. (canceled)

19. A method of identifying a strain or serovar of Salmonella enterica in a sample comprising:
a) determining an allele corresponding to a single nucleotide polymorphism (SNP) for a SNP panel comprising fifty-two SNPs for nucleic acids isolated from the sample comprising the steps of:
(i) identifying at least a first target nucleic acid sequence comprising a first SNP from the panel of fifty two SNPs wherein the first SNP is comprised at position 101 in nucleic acid sequences described in SEQ ID NO: 1-SEQ ID NO: 52 or complementary sequences thereof;
(ii) hybridizing at least a first pair of polynucleotide primers to the first target nucleic acid sequence comprising a first SNP;
(iii) amplifying the first target nucleic acid sequence to form a first amplified target nucleic acid sequence product comprising the first SNP;
(iv) determining the allelic composition of the first SNP from the first amplified target nucleic acid sequence product comprising the first SNP;
(v) repeating steps (i)-(iv) using a different set of primer pairs, each primer pair operable to hybridize to and amplify a target nucleic acid sequence comprising another SNP and determining the allelic composition of each SNP until the allelic compositions of the fifty-two SNP panel are determined.
b) creating a SNP profile of the sample nucleic acids for which the allelic compositions are determined;
c) comparing the SNP profile of the sample nucleic acids with a master SNP profile database; and
d) correlating the SNP profile of the sample nucleic acids with the master SNP profile database to determine the strain or serovar of the Salmonella enterica comprised in the sample, wherein the presence of certain alleles in the SNP profile of the sample nucleic acids that correspond to a strain or serovar of the master SNP profile database determines the strain or serovar of Salmonella enterica in the sample.

20. The method of claim 19, wherein the master SNP profile database is stored on a computer readable medium, and wherein the step of comparing and the step of correlation of SNP profiles of the sample nucleic acids with the master SNP profile database are performed using a computer system or are performed manually.

21. The method of claim 19, wherein the master SNP profile database comprises the compositions of all known SNP profiles for each SNP position and comprises the correlation of the SNP allelic composition and position with different Salmonella enterica serovars or strains

22. The method of claim 19, wherein the master SNP profile database comprises at least fifty two SNPs comprised at position 101 in nucleic acid sequences described in SEQ ID NO: 1 - SEQ ID NO: 52 or complementary sequences thereof.

23. The method of claim 19, wherein the master SNP profile database comprises all known SNP profiles of Salmonella enterica serovars or strains including the at least fifty two SNPs comprised at position 101 in nucleic acid sequences described in SEQ ID NO: 1-SEQ ID NO: 52 or complementary sequences thereof.

24. The method of claim 19, wherein amplification is selected from the group consisting of polymerase chain reaction (PCR), RT-PCR, quantitative PCR, asynchronous PCR (A-PCR), and asymmetric PCR (AM-PCR), strand displacement amplification (SDA), multiple displacement amplification (MDA), nucleic acid strand-based amplification (NASBA), rolling circle amplification (RCA), transcription-mediated amplification (TMA).

25. The method of claim 19 wherein the polynucleotide primer pairs are selected from polynucleotides having the sequence of SEQ ID NO: 54-SEQ ID NO: 157, complements thereof, and labeled derivatives thereof.

26. The method of claim 19, wherein determining the allelic composition of each SNP from the panel of fifty-two SNPs comprises analyzing the amplification product by size analysis; sequencing; hybridization; 5′ nuclease digestion; single-stranded conformation polymorphism; allele specific hybridization; primer specific extension; oligonucleotide ligation assay and combinations thereof.

27. The method of claim 19, wherein the determining the allelic composition of each SNP of the panel of at least fifty-two SNPs comprises analyzing the amplification product by 5′ nuclease digestion using a labeled probe for each allele of the SNP.

28. The method of claim 27, wherein the labeled probe has one label.

29. The method of claim 27, wherein the labeled probe has two labels.

30. The method of claim 29, wherein one label is a fluorescent reporter and the second label is a quencher.

31. The method of claim 27, wherein the labeled probe is a nuclease probe.

32. The method of claim 27, wherein the labeled probe is selected from the polynucleotides having SEQ ID NO: 158-261.

33. The method of claim 26, wherein determining the allelic composition comprises hybridization and comprises the steps of:
a) providing at least a first labeled probe comprising an isolated polynucleotide sequence operable to bind to the first SNP having a first allelic composition of the first SNP;
b) contacting the-sample nucleic acid with the first labeled probe under conditions suitable for hybridization;
c) detecting hybridization of the sample nucleic acid with the first labeled probe, wherein the detection of at least one hybridized nucleic acid is indicative of the presence of the first SNP having a first allelic composition of the first SNP in the sample nucleic acid;
d) repeating the steps a)-c) for each of the second allele of the first SNP using a second labeled probe for the second allele; and
repeating steps a)-d) for each allele of the fifty-two SNP panel.

34. The method of claim 33, wherein the first and the second labeled probes for each allele have a different label.

35. The method of claim 19, wherein determining the allelic composition of the first SNP from the first amplified target nucleic acid sequence product comprises:
a) providing at least a first probe comprising an isolated polynucleotide sequence operable to bind to the first SNP wherein the first SNP has a first allelic composition;
b) providing at least a second probe comprising an isolated polynucleotide sequence operable to bind to the first SNP wherein the first SNP has a second allelic composition;
c) contacting the isolated sample nucleic acid with the first probe and the second probe under conditions suitable for hybridization; and
d) detecting hybridization of the sample nucleic acid with either the first probe or the second probe, wherein the detection of a hybridized nucleic acid comprising the first probe is indicative of the presence of the first SNP having the first allelic composition and wherein the detection of a hybridized nucleic acid comprising the second probe is indicative of the presence of the first SNP having the second allelic composition,
thereby determining if the allelic composition of the first SNP in a sample nucleic acid corresponds to the first allelic composition or the second allelic composition.

36. The method of claim 35, wherein the first probe is labeled with a first detectable label and the second probe is labeled with a second detectable label.

37. The method of claim 36, wherein the first probe comprises an isolated polynucleotide sequence selected from SEQ ID NO: 158-SEQ ID NO: 209, and a second probe comprising an isolated nucleotide sequence selected from the group consisting of SEQ ID NO: 210-SEQ ID NO: 261.

38. The method of claim 19, wherein the primer nucleotide sequences comprise at least 15 contiguous nucleotides of any one of SEQ ID NO: 1-SEQ ID NO: 52 that flank at least the nucleotides located at positions 100-103 of SEQ ID NO: 1-SEQ ID NO: 52.

39. The method of claim 27, wherein the probes comprise at least 15 contiguous nucleotides of any one of SEQ ID NO: 1-SEQ ID NO: 52 that comprise at least the nucleotides located at positions 100-103 of SEQ ID NO: 1-SEQ ID NO: 52 and a label.

40. A kit to detect a strain or serovar of Salmonella enterica in a sample comprising:
polynucleotide primer pairs having the sequence of SEQ ID NO: 54-SEQ ID NO: 157, complements thereof, and labeled derivatives thereof;
probes having labeled derivatives of polynucleotides having SEQ ID NO: 158-SEQ ID NO: 261; and
one or more components selected from buffers, enzymes, nucleotides, salts, and combinations thereof.
